(19) United States
(12) Patent Application Publication
(10) Pub. No.: US 2023/0226047 A1
(43) Pub. Date: Jul. 20, 2023

(54) COMBINATION THERAPY FOR TREATMENT OF CANCERS

(71) Applicant: Health Research, Inc., Buffalo, NY (US)

(72) Inventor: Sebastiano BATTAGLIA, Buffalo, NY (US)

(21) Appl. No.: 17/768,455

(22) PCT Filed: Oct. 16, 2020
(86) PCT No.: PCT/US2020/056150

§ 371 Date: Apr. 12, 2022

Related U.S. Application Data
(60) Provisional application No. 62/916,025, filed on Oct. 16, 2019.

(51) Int. Cl. A61K 31/498; A61P 35/00; A61K 39/395

(52) U.S. Cl. CPC A61K 31/498; A61P 35/00; A61K 39/3955

(57) ABSTRACT

Provided are methods and compositions for the treatment of cancer. The methods comprise administering to an individual in need of treatment inhibitors of WHSC1 expression, function or activity in combination with PARP inhibitors or immune based therapy. In an aspect, the present disclosure provides compositions comprising one or more WHSC1 inhibitors and one or more PARP inhibitors, or one or more WHSC1 inhibitors and one or more immune checkpoint inhibitors.

### CROSS-REFERENCE TO RELATED APPLICATIONS

This application claims priority to U.S. Provisional patent application no. 62/916,025, filed on Oct. 16, 2019, the disclosure of which is incorporated herein by reference.

### BACKGROUND OF THE DISCLOSURE

Prostate cancer (PCa) is the most commonly diagnosed cancer in men in the US. Overcoming diagnosis and treatment for PCa can have a significant ripple effect on patients and their families. Moreover, the cost of treatment increases exponentially when patients fail to respond and develop metastatic disease concomitantly with adverse side effects, further indicating that being able to offer alternative therapeutic approaches with fewer side effect and increased anti-cancer potency would reduce the burden on patients and their families. The prostate gland has for long been considered an immune privileged organ, and the role of the immune system in mediating PCa growth and therapeutic response has been often underestimated. However, tumor cells can escape the immune system not only through physical and molecular barriers, but also through coordinated processes aimed to repress antigen processing machinery (APM) genes, downregulate major histocompatibility complex(es) (MHC) and upregulate checkpoint proteins. Such processes limit the ability of immune cells to reach the tumor site, recognize tumor cells and mount a potent anti-tumor immune response. This paradigm has been challenged by studies targeting the prostate specific antigen (PSA) or the prostate acid phosphatases (PAP), which demonstrate that immune cells can populate the prostate and re-activate immune moieties against PCa cells. Albeit the initial promising data, recent trials testing the efficacy of checkpoint blockade therapy in patients with recurrent PCa report negative and contrasting results. These results indicate a strong need to develop therapeutic regimens that address and overcome tumor immune evasion in PCa and other cancers to improve upon current treatment options.

### SUMMARY OF THE DISCLOSURE

This disclosure describes the role of Wolf-Hirschhorn syndrome candidate gene-1 (WHSC1) in the context of immune evasion and in the context of modifying BRCA status. The WHSC1/nuclear set domain containing 2 (NSD2) gene encodes for an epigenetic enzyme, a histone methyltransferase, which targets H3K36me2/me3, and to a minor extent, H3K20me2. High levels of WHSC1 correlate with worse prognosis, development of metastases and resistance to chemotherapy. Using publicly available patient data and complementary human and murine cell line models, we demonstrate computationally, in vitro and in vivo that WHSC1 negatively correlates with the expression of genes in the antigen processing and presentation machinery (APM) pathways. In vivo study demonstrates further the relevance of the immune system in aiding the anti-tumor effect of WHSC1 inhibition by showing reduced tumor growth, increased MHC expression and immune infiltration in immunocompetent mice, but not in immunocompromised mice. Lastly, WHSC1 inhibition downregulates genes (cyclin dependent kinase 12 (CDK12), breast cancer gene (BRCA1/2), melanocyte stimulating hormone (MSH) genes and poly-ADP ribose polymerase (PARP)) whose inactivation was previously associated with response to checkpoint blockade.

Based at least in part on these findings, the present disclosure provides compositions and methods for the treatment of prostate cancer and other cancers, with no limitation in terms of tissue of origin. The methods comprise administering to an individual in need of treatment a combination therapy comprising inhibition of WHSC1 expression or protein (such as by using an inhibitor or inhibition may be carried out by using clusters of regularly interspaced short palindromic repeats (CRISPR)), and PARP inhibitor (PARPi) or immune based therapy, or other molecules identified by screening methods such as, but not limited to, next generation sequencing (NGS), companion diagnostic, immunohistochemistry or other methods. In an embodiment, the combination therapy comprises a WHSC1 inhibitor, and a PARP inhibitor and/or immune based therapy.

In an aspect, the present disclosure provides compositions comprising one or more WHSC1 inhibitors and one or more PARP inhibitors, or one or more WHSC1 inhibitors and one or more immune checkpoint inhibitors.

### BRIEF DESCRIPTION OF THE FIGURES

FIG. 1. Kaplan Meier plot showing disease free survival comparing patients with high vs. low expression levels of WHSC1 using the TCGA PCa cohort, shown in A). Patients were divided based on the top/bottom 25% gene expression, Cox HR, p.value and median survival were calculated for the two groups and showed on the plot. B) Predicted infiltration levels of immune cells in TCGA PCa data using pre-computed data from xCell. HIGH and LOW refer to patients with the top/bottom 25% of WHSC1 expression. P.value showed in figure, calculated with two-tailed Student's t-test, n=125/group. C) Heatmap showing HLA expression in the TCGA PCa cohorts. The right bar block and the left bar block for each indicate up- and down-regulated genes, respectively. Side annotation indicates the patients groups based on WHSC1 expression levels. D-E) GSEA analysis comparing patients with high vs. low WHSC1 levels highlighting upregulated (D) and downregulated (E) pathways in patients with elevated WHSC1 expression levels.

FIG. 2. Heatmap of DEGs within H3K36me3 loci identified from TCGA data. Red and green indicate high and low expression levels, respectively. Blue and red annotation indicate patients with high and low WHSC1 expression, respectively. Black arrows indicate selected genes (DNMT1, DNMT3A, CD274, B2M, HLA-C and WHSC1).

FIG. 3. RNASeq analysis in C42 cells following shRNA knockdown of WHSC1, shown in A). Dark grey dots indicate genes with FDR<=0.05. B) Boxplots showing the expression levels of AR and KLK2 following WHSC1 knock down, p value on figure calculated with limma, n=3/group. C) Gene Set Enrichment Analysis (GSEA) indicating pathways down- and up-regulated following knockdown of WHSC1. D) GSEA analysis using a custom APM-MHC gene signature. E) Heatmap showing expression of

HLAs, DNA repair genes and DNMT1 in C42 comparing knockdown vs. control. Red and blue cells indicate high and low expression levels, respectively. F) Expression of a panel of TAP processing genes, mostly upregulated, in RNASeq data using C42 knockdown cells. Red and black cells indicate high and low expression levels, respectively. G) RNASeq data from shNSD2 C42 cells showing the expression of TAP processing genes and DNMT1 present within the H3K36me2 mark from GSM225904. Purple and green cells indicate high and low expression levels, respectively.

FIG. 4. Distribution of the beta values in our methylomic analysis, shown in A). PCA analysis of the normalized methylation data, values were scaled and centered prior PCA analysis, shown in B). Blue and red dots indicate controls (shCTR) and knockdown (shWHSC1) samples, respectively. C) Scatterplot showing the relationship between the changes in percentage methylation and gene expression following WHSC1 knockdown. Dark grey dots highlight genes with negative correlation (high methylation, low expression and vice versa) that were selected. D) Heatmap visualization of the methylation intensity vs. expression values, highlighting genes that belong to immune and APM pathways. For methylation data (left), orange and blue indicate high and low intensity/beta values, respectively. For RNASeq data, red and cyan indicate high and low gene expression, respectively.

FIG. 5. ATAC Seq log2FC obtained by calculating the difference in read abundance from common loci in control and knock down cells, shown in A). B) Summary of the log2FC vs. fragment length, with the dotted vertical lines indicating the periodicity of the nucleosomes. C) Correlation between log2FC in ATACSeq and RNASeq data, each dot is a gene, highlighted in dark grey are genes showing positive correlation between the two datasets. D-E) GSEA analysis using the abovementioned data. F) Boxplots indicating gene expression in the genes involved in the upregulated pathways (n=3/group, p value on figure, calculated with limma). G) ATACSeq reads for representative genes confirming increased peaks in boxes following knockdown of WHSC1.

FIG. 6. Protein data from prostates isolated from WT and TRAMP mice at different stages of PCa development testing the protein levels for WHSC1, DNMT1, CD274 and H3K36me2, shown in A). B) qPCR validation for DNMT1 and CD274 following WHSC1 knockdown. (n=3/group, two tailed Student's t-test, *, p<0.05)

FIG. 7. Flow cytometry (A-B) analysis of MHC expression levels upon pharmacological WHSC1 inhibition and IFNg treatment in human C42 (A) and murine C2

(B) cells (n=3/group, one-way ANOVA with post hoc Tukey correction. *,p<0.05, **,p<0.01, ***,p<0.001). C) Flow plot showing increased H2Kb-bound to OVA following treatment with MCTP39, and (D) its quantification.

FIG. 8. Growth curve of TRAMP C2 cells grafted in C57B/6 mice following treatment with MCTP39 for four weeks, shown in A). Lines indicate growth in control and MCTP39-treated mice, respectively. n=6/group, p=0.0023, permutation test. B) Growth curve of TRAMP C2 cells in NSG mice following four weeks treatment with MCTP39. Black and gray lines indicate growth in control and MCTP39 treated mice, respectively. p=0.1461, NS (Not Significant), permutation test. C) Quantification of flow cytometry data in tumors at endpoint evaluating, from left to right, CD8⁺ T cells infiltration, H2Kb expression on the tumor and tumor weight in grams in C56B/6 mice. n=6/group, two-tailed Student's t-test *,p<0.05, **,p<0.01.

FIG. 9. Area under the curve (AUC) analysis inferring the value of WHSC1 in predicting biochemical recurrence, shown in A). B) Distribution of 10,000 random AUCs calculated using TCGA RNASeq data, compared to the observed AUC for WHSC1 (line). C) Correlation of WHSC1 expression (x-axis) with the AR expression (y-axis) using RNASeq data from TCGA.

FIG. 10. Panel A) shows C42 cells were stably transfected with either shRNA targeting WHSC1 or shCTR (bottom). Cell proliferation was monitored (top) and reported as ratio to the control after 96 hours. Experiments were repeated in biological triplicate. **, p<0.01, ANOVA with post hoc Tukey correction, n=3/group. B) DU145 cells were transiently transfected with either scrambled control (siCTR) or siWHSC1 and measured via qRT-PCR (top)(Two-tailed Student's t-test, n=2, p=0.01). Cell viability was measured by MTT after 96 h (bottom)(Two-tailed Student's t-test, n=3, p=0.05).

FIG. 11. Panel A) shows siRNA knockdown of WHSC1 in TRAMP C2 cells and B) cell proliferation at 96 hours post transfection (n=3/group, Student's t-test, p=0.02)

FIG. 12. Table S1 showing characteristics of the TCGA prostate cancer cohort used in this study.

FIG. 13. Table S2 showing primers used.

FIG. 14. Table S3 showing antibodies used.

FIG. 15. Pan cancer overall survival analysis comparing top vs. bottom 25% WHSC1 expressing samples.

FIG. 16. GSEA analysis comparing HIGH vs. LOW group showing upregulation of proliferative and DNA repair pathways and downregulation of immune pathways, shown in A). B) Log2FC of HLA genes, WHSC1 (gray) and DNA repair genes (remaining) comparing HIGH vs. LOW groups.

FIG. 17. Ranked expression of WHSC1 across TCGA lung cancer patients, shown in A), survival analysis comparing lung cancer patients with high (red) WHSC1 and low (blue) WHSC1 mRNA levels, shown in B). C) Number of mutation in patients with high WHSC1 and low WHSC1 mRNA levels. D) Mutations in key lung cancer genes in patients with for each set high (left) WHSC1 and low (right) WHSC1 mRNA levels. E) Genome wide correlation analysis of all genes against WHSC1. x-axis indicates the Log2FC, y-axisindicates the −log10(qValue). F) Levels of CD3E, CD274/PDL1 and IL2 in patients with high WHSC1 and loWHSC1 mRNA levels.

FIG. 18. GSEA analysis of RNASeq data following shRNA knockdown of WHSC1 in C42 cells, shown in A). Pathway name is indicated followed by p value and normalized enrichment score (NES). Positive and negative NES is indicative of upregulated and downregulated pathway, respectively. B) Heatmap from RNASeq data showing the expression of HLAs, DNA repair genes, and immune-related genes in C42 cells comparing shWHSC1 vs. control. Red and blue cells indicate high and low expression levels, respectively.

FIG. 19. Network analysis of BRCA-related genes. Interaction data were obtained from Pathway Common and further analyzed in R. Blue and red nodes indicate down- and up-regulated genes, respectively. Green lines indicate physical interaction/same complex, while orange line indicate regulatory interactions (e.g. transcriptional regulation).

FIG. 20. MHC expression on intratumoral CD11c⁺ DCs in the control (left) and MCTP39-treated mice (right).

FIG. 21. Gene expression of genes involved with DNA repair (MSH2, RAD51).

FIG. 22. Gene expression of 9 genes involved with response to anti-programmed cell death protein 1 (PD1) therapy. Data from RNASeq data in C42 shWHSC1 cells compared to control. KD=shWHSC1, WT=control.

FIG. 23. Timing for tumor growth and treatment.

### DESCRIPTION OF THE DISCLOSURE

The present disclosure provides compositions and methods for treatment of cancers. To determine if WHSC1 association with MHC expression and DNA repair is present in various cancer types, we mined the TCGA Pan-Cancer RNASeq data, dividing patients in the top vs. bottom 25% expression for WHSC1. Consistent with our preliminary data on PCa, patients with high WHSC1 have significantly shorter overall survival, irrespective from tumor type. After removing samples with low variance, we performed GSEA analysis comparing high vs. low WHSC1 groups, irrespective of tumor type. Results show a pattern strikingly consistent with our preliminary data of significant upregulation of DNA repair and proliferative pathways, and downregulation of antigen processing, response to IFN gamma and cytokines. Moreover, at the gene level, there is a net downregulation of HLA molecules and upregulation of genes involved with DNA damage repair in patients with elevated WHSC1.

Throughout this application, the use of the singular form encompasses the plural form and vice versa. For example, “a”, or “an” also includes a plurality of the referenced items, unless otherwise indicated.

Where a range of values is provided in this disclosure, it should be understood that each intervening value, and all intervening ranges, between the upper and lower limit of that range is also included, unless clearly indicated otherwise. The upper and lower limits from within the broad range may independently be included in the smaller ranges encompassed within the disclosure.

The term “therapeutically effective amount” as used herein refers to an amount of an agent sufficient to achieve, in a single or multiple doses, the intended purpose of treatment. Treatment does not have to lead to complete cure, although it may. Treatment can mean alleviation of one or more of the symptoms or markers of the indication. The exact amount desired or required will vary depending on the particular compound or composition used, its mode of administration, patient specifics and the like. Appropriate effective amount can be determined by one of ordinary skill in the art informed by the instant disclosure using only routine experimentation. Within the meaning of the disclosure, “treatment” also includes prophylaxis and treatment of relapse, as well as the alleviation of acute or chronic signs, symptoms and/or malfunctions associated with the indication. Treatment can be orientated symptomatically, for example, to suppress symptoms. It can be effected over a short period, over a medium term, or can be a long-term treatment, such as, for example within the context of a maintenance therapy. Administrations may be intermittent, periodic, or continuous.

The present disclosure is based on the surprising identification of the role of WHSC1 in the context of immune evasion. Using patients data and complementary human and murine cell line models, we identified, in vitro and in vivo that WHSC1 negatively correlates with the expression of genes in the antigen processing and presentation machinery (APM) pathways. In vivo study demonstrated further the relevance of the immune system in aiding the anti-tumor effect of WHSC1 inhibition by showing reduced tumor growth, increased MHC expression and immune infiltration in immunocompetent mice, but not in immunocompromised mice. Further, WHSC1 inhibition downregulates genes (CDK12, BRCA1/2, MSH genes and PARP) whose inactivation is known to be associated with response to checkpoint blockade. Based at least on these observations, the present method provides a method for inhibition of growth of cancer cells by a combination therapy comprising inhibition of WHSC1 and immune based therapies.

In an aspect, this disclosure provides compositions for treatment of cancers. In embodiments, the compositions achieve inhibition of WHSC1 expression, function or activity in combination with inhibition of PARP and/or in combination with immune based therapy. In various embodiments, the compositions comprise combinations of one or more WHSC1 inhibitors and one or more PARP inhibitors, combinations of one or more WHSC1 inhibitors and one or more immune checkpoint inhibitors, or combinations of one or more WHSC1 inhibitors and dendritic cell (DC) vaccines, combination of one or more WHSC1 inhibitors and DNMT1 inhibitors, combination of one or more WHSC1 inhibitors and EZH2 inhibitors, combination of one or more WHSC1 inhibitors and AKT inhibitors, combination of one or more WHSC1 inhibitors and MTOR inhibitors, combination of one or more WHSC1 inhibitors and BCL2 inhibitors, combination of one or more WHSC1 inhibitors and ER-targeting molecules, combination of one or more WHSC1 inhibitors and AR-targeting molecules, combination of one or more WHSC1 inhibitors and VEGF inhibitors, combination of one or more WHSC1 inhibitors and EGFR inhibitors, combination of one or more WHSC1 inhibitors and TKIs, combination of one or more WHSC1 inhibitors and aromatase inhibitors, combination of one or more WHSC1 inhibitors and DNA based therapies, combination of one or more WHSC1 inhibitors and small molecules targeting genes interacting with WHSC1.

WHSC1 inhibition may be carried out by using inhibitors of WHSC1 (also referred to herein as WHSC1/NSD2). An example of a WHSC1 inhibitor is MCTP-39. Other examples include LEM-14 (PMID: 30471851), DZNep, DA3003-1, Chaetocin, ABT-199, PF-03882845, TC LPA5 4 (PMID: 29945974).

The disclosure includes disrupting the target gene such that WHSC1 mRNA and protein are not expressed. For example, the WHSC1 gene (Genbank ID: AF083386) can be disrupted by targeted mutagenesis. The sequences and all variants thereof of any sequences referenced herein are incorporated herein by reference as of the filing date of this application. In embodiments, targeted mutagenesis can be achieved by, for example, targeting a CRISPR site in the target gene. So-called CRISPR systems designed for targeting specific genomic sequences are known in the art and can be adapted to disrupt the target gene for making modified cells encompassed by this disclosure. In general, the CRISPR system includes one or more expression vectors encoding at least a targeting RNA and a polynucleotide sequence encoding a CRISPR-associated nuclease, such as CRISPR associated protein (Cas) 9, but other Cas nucleases can alternatively be used. CRISPR systems for targeted disruption of mammalian chromosomal sequences are commercially available.

Examples of PARP inhibitors useful for the present methods include, but are not limited to, NU1025; 3-aminobenzamide; 4-amino-1,8-naphthalimide; 1,5-isoquinolinediol; 6(5H)-phenanthriddinone; 1,3,4,5,-tetrahydrobenzo(c)(1,6)- and (c)(1,7)-naphthyridin-6 ones; adenosine substituted 2,3-dihydro-1H-isoindol-1-ones; AG14361; AG014699; 2-(4-chlorophenyl)-5-quinoxalinecarboxamide; 5-chloro-2-[3-(4-phenyl-3,6-dihydro-1 (2H)-pyridinyl)propyl]-4(3H)-quinazolinone; isoindolinone derivative INO-1001; 4-hydroxyquinazoline; 2-[3-[4-(4-chlorophenyl) 1-piperazinyl]propyl]-4-3(4)-quinazolinone; 1,5-dihydroxyisoquinoline (DHIQ); 3,4-dihydro-5[4-(1-piperidinyl)(butoxy)-1(2H)-isoquinolone; CEP-6800; GB-15427; PJ34; DPQ; BS-201; AZD2281 (Olaparib); BS401; CHP101; CHP102; INH2BP; BSI201; BSI401; TIQ-A; an imidazobenzodiazepine; 8-hydroxy-2-methylquinazolinone (NU1025), CEP 9722, MK 4827, LT-673; 3-aminobenzamide; Olaparib (AZD2281; ABT-888 (Veliparib); BSI-201 (Iniparib); Rucaparib (AG-014699); A-966492; PJ-34; and talazoparib. In an embodiment, the PARPi may be olaparib, rucaparib, niraparib, talazoparib, veliparib, pamiparib, CEP9722, E7016, 3-Aminobenzamide or combinations thereof.

Immune based therapies that may be used in the combination therapy (e.g., in combination with WHSC1/NSD2 inhibitors), include immune checkpoint inhibitors (e.g., anti-PD-1, anti-PD-L1, anti-cytotoxic T lymphocyte-associated protein 4 (CTLA-4), anti-lymphocyte activation gene 3 (LAG3) etc.), which may be small molecule inhibitors or monoclonal antibodies, vaccines (e.g., dendritic cell-based; viral-based; autologous whole tumor cell), adoptive cellular therapy (e.g., tumor infiltrating lymphocytes (TILs); T cell receptor-engineered lymphocytes; chimeric antigen receptor (CAR) T cells or CAR natural killer (NK) cells).

Immune checkpoint inhibitors may include targeting one or more immune checkpoints, including, but not limited to, PD-1/PD-L1, CTLA-4, LAG-3, OX40, T cell immunoglobulin domain and mucin domain 3 (TIM-3) and B7-H3. PD-1/PD-L1 and TIM-3 suppress normal T-cell activation and function. PD-1 is a T-cell surface receptor that is expressed on T cells, B cells, NK cells, activated monocytes and dendritic cells. The role of PD-1 in normal human physiology is to limit autoimmunity by acting as a co-inhibitory immune checkpoint expressed on the surface of T cells and other immune cells, including tumor-infiltrating lymphocytes. It has two ligands: PD-L1/B7-H1 and PD-L2/B7-DC. CTLA-4 and B7-H3 are considered to inhibit T-cell function and become overexpressed in most solid cancers such as breast cancer, prostate cancer, renal cell carcinoma, liver cancer and brain cancer. LAG-3 is a surface molecule that promotes activation of T-cells. OX40 is a surface molecule in the tumor necrosis factor receptor family.

Monoclonal antibodies against immune checkpoints include antibody therapies directed against immune checkpoints PD-1 (e.g., nivolumab, pembrolizumab, cemiplimab, pidilizumab, duralumab), PD-L1 (e.g., atezolizumab, durvalumab, avelumab), CTLA-4 (e.g., ipilimumab, tremelimumab), and immune-activating antibodies (e.g., directed against 41BB (e.g., utomilumab).

Several small molecules are known to inhibit various immune checkpoints. Small molecule inhibitors (SMI) that affect PD-1/PD-L1, include BMS-8, BMS-37, BMS-202, BMS-230, BMS-242, BMS-1001 and BMS-1166, SB415286, vorinostat, panobinostat, azacitidine, decitabine, entitostat, JQ1, I-BET151, GSK503. SMIs that affect CTLA4 include entitostat, panobinostat, ACY-241, azacytidine. SMIs that affect OXO include PF-04518600, ABBV-368, DB36, DB71, DB15, CVN, MGCD0103, SNDX-275, azacytidine. Small molecule inhibitors that affect LAG-3 include TSR-033, IMP32, BMS986016. Small molecule inhibitors that affect TIM-3 include TSR-022, Sym023, ATIK2a, and SMIs that affect B7-H3 include c-MYC SMIs, vorinostat, DZNep.

Examples of T cell-based immunotherapies include adoptive cell transfer therapies in which patients are infused with their own immune cells (e.g., T cells include enriched populations of tumor-reactive T cells, genetically-engineered CAR-T cells (chimeric antigen receptor T cells) or T cell receptor-engineered T cells, and natural killer cells (NK cells; FATE-NK100)).

Cancer vaccines include vaccines based on tumor cells, tumor lysates or tumor associated antigens, and dendritic cell (DC)-based vaccines.

Generally, a therapeutically effective amount of an antibody, small molecules, or other compounds or compositions described herein can be in the range of 0.01 mg/kg to 100 mg/kg and all values therebetween. For example, it can be 0.1 mg/kg to 100 mg/kg, 0.1 mg/kg to 50 mg/kg, 1 mg/kg to 50 mg/kg etc.

The WHSC1/NSD2 inhibitor(s) and PARP inhibitor(s) or the WHSC1/NSD2 inhibitor(s) and the immune therapy (e.g., checkpoint inhibitor) may be administered in separate compositions or in the same composition, via the same route or separate routes, over a same period of time or different periods of time. The two administrations regimens may overlap partially or completely or not at all. The compositions may comprise a pharmaceutically acceptable carrier or excipient, which typically does not produce an adverse, allergic or undesirable reaction when administered to an individual, such as a human subject. Pharmaceutically acceptable carrier or excipient may be fillers (solids, liquids, semi-solids), diluents, encapsulating materials and the like. Examples include, but are not limited to, saline, buffered saline, dextrose, water, glycerol, ethanol, etc.

The pharmaceutical compositions may be in the form of solutions, suspensions, emulsions, and solid injectable compositions that are dissolved or suspended in a solvent immediately before use. The injections may be prepared by dissolving, suspending or emulsifying one or more of the active ingredients in a diluent. Examples of diluents are distilled water for injection, physiological saline, physiologic buffer, vegetable oil, alcohol, and a combination thereof. Further, the compositions may contain stabilizers, solubilizers, suspending agents, emulsifiers, soothing agents, buffers, preservatives, etc. The pharmaceutical compositions may be formulated into a sterile solid or powdered preparation, for example, by freeze-drying, and may be used after sterilized or dissolved in sterile injectable water or other sterile diluent(s) immediately before use. The compositions can include one or more standard pharmaceutically acceptable carriers. Some examples herein of pharmaceutically acceptable carriers can be found in: Remington: The Science and Practice of Pharmacy (2013) 22nd Edition, Pharmaceutical Press.

The pharmaceutical compositions of the invention may be administered via any route that is appropriate, including but not limited to oral, parenteral, sublingual, transdermal, rectal, transmucosal, topical, via inhalation, via buccal administration, or combinations thereof. Parenteral administration includes, but is not limited to, intravenous, intraarterial, intraperitoneal, subcutaneous, intratumoral, intramuscular, intrathecal, and intraarticular. The agents(s) can also be administered in the form of an implant, which allows a slow release of the compound(s), as well as a slow controlled i.v. infusion. The

WHSC1/NSD2 inhibitors and PARP inhibitors, or WHSC1/NSD2 inhibitors and immune therapy may be delivered via different routes or the same routes.

In an aspect, this disclosure provides methods for the treatment of cancer. The methods comprise inhibiting WHSC1 expression, function or activity in combination with inhibition of PARP and/or immune based therapy. In an embodiment, the method comprises administering to an individual one or more WHSC1 inhibitors and one or more PARP inhibitors. The one or more WHSC1 inhibitors and one or more PARP inhibitors may be administered concurrently or sequentially or in overlapping regimens, and via the same route or different routes. In an embodiment, the method comprises administering to an individual one or more WHSC1 inhibitors and one or more immune checkpoint inhibitors. The one or more WHSC1 inhibitors and one or more immune checkpoint inhibitors may be administered concurrently or sequentially or in overlapping regimens, and via the same route or different routes. In an embodiment, the method comprises administering to an individual one or more WHSC1 inhibitors and cancer vaccine. The one or more WHSC1 inhibitors and cancer vaccine may be administered concurrently or sequentially or in overlapping regimens, and via the same route or different routes. In an embodiment, the method comprises administering to an individual one or more WHSC1 inhibitors and one or more small molecules targeting a gene interacting with, controlled by, or controlling the expression of WHSC1. The one or more WHSC1 inhibitors and one or more small molecules may be administered concurrently or sequentially or in overlapping regimens, and via the same route or different routes. The length of the treatment with WSCH1 inhibitor alone or in combination with other molecules is dictated by the specific clinical circumstances of the patient.

Individuals who may receive the combination treatment described herein include those afflicted with or diagnosed with a cancer. Examples include but are not limited to, prostate cancer, testicular cancer, pancreatic cancer, lung cancer, which may be non-small cell lung cancer (NSCLC), which may be squamous cell (or epidermoid) carcinoma, adenocarcinoma and, large cell (or undifferentiated) carcinoma, or any other type, melanoma of the skin, kidney cancer, bladder cancer, liver cancer, colon cancer, head and neck cancers, breast cancer, ovarian cancer, cervical cancer, Hodgkin lymphoma, urinary tract cancers, and other types of cancers. The cancer, such as lung cancer or breast cancer may be refractory to current treatments. The breast cancer may be metastatic triple-negative breast cancer, all stages, and may be refractory to current treatments. Individuals who may receive the present combination therapy may include those who have already undergone other types of therapies, including chemotherapy, surgical intervention (including removal of tumor mass or affected organs, such as in castration), or hormonal therapy and the like.

The WHSC1/NSD2 inhibitor and the immune therapy may be administered concurrently or sequentially. For example, the WHSC1/NSD2 inhibitor regimen may be administered first and then after a suitable period of time, the immune therapy regimen may be started. Their administration may overlap. Alternatively, they may be administered in the reverse order. For example, the immune therapy regimen may be administered first and then after a suitable period of time, the WHSC1/NSD2 inhibitor regimen may be started. Their administration may overlap. Similarly, WHSC1/NSD2 inhibitor and the PARP inhibitor may be administered concurrently or sequentially. For example, the WHSC1/NSD2 inhibitor regimen may be administered first and then after a suitable period of time, the PARP inhibitor regimen may be started. Their administration may overlap. Alternatively, they may be administered in the reverse order. For example, the PARP inhibitor regimen may be administered first and then after a suitable period of time, the WHSC1/NSD2 inhibitor regimen may be started. Their administration may overlap. Similarly, WHSC1/NSD2 inhibitor and the small molecules may be administered concurrently or sequentially. For example, the WHSC1/NSD2 inhibitor regimen may be administered first and then after a suitable period of time, the small molecules regimen may be started. Their administration may overlap. Alternatively, they may be administered in the reverse order. For example, the small molecules regimen may be administered first and then after a suitable period of time, the WHSC1/NSD2 inhibitor regimen may be started. Their administration may overlap.

In an aspect, this disclosure provides kits for the treatment of cancer. The kit may comprise in a single or separate compositions: i) one or more of WHSC1/NSD2 inhibitors and ii) PARP inhibitors or immune checkpoint inhibitors. Optionally, buffers and instructions for administration may also be provided. In an embodiment, the disclosure provides a kit comprising in separate sterile containers, one or more doses of a WHSC1/NSD2 inhibitor and a PARP inhibitor, and optionally, instructions for use and diluting buffers or solutions. In an embodiment, the disclosure provides a kit comprising in separate sterile containers, one or more doses of a WHSC1/NSD2 inhibitor and an immune checkpoint inhibitor, and optionally, instructions for use and diluting buffers or solutions.

The following example is provided to illustrate the invention and is not intended to be restrictive.

### EXAMPLE 1

This example demonstrates a novel role for WHSC1 in defining immune infiltration in PCa, with significant future implications for the use of immunotherapies in prostate malignancies.

Immunotherapy in prostate cancer (PCa) lags behind the progresses obtained in other cancer types partially because of its limited immune infiltration. While a few studies reported increased homing of immune cells in response to androgen deprivation therapy (ADT), the mechanism by which this occurs is still poorly understood. Using TCGA PCa RNASeq data patients were divided into top/bottom 30% based on the expression of WHSC1 and DFS was calculated for both groups. Publicly available ChIPSeq data were obtained from Cistrome and integrated with the available RNASeq data. RNASeq, ATACSeq and EPIC Infinium MethylArrays were analyzed using R Bioconductor packages comparing C42 cells with or without stable knockdown on WHSC1. Flow cytometry was used to measure MHC levels, MHC-bound OVA and tumor infiltration. C57B6 and NSG mice were subcutaneously grafted with TRAMP C2 cells and treated with MCTP39 (10 mg/kg); tumor size was monitored over time and curves compared using permutation analyses. All analyses use a significance threshold of 0.05.

Leveraging TCGA data we demonstrate that elevated WHSC1 levels positively correlate with the presence of an immunosuppressive microenvironment. We validated those results in vitro, demonstrating that genetic and pharmacological inhibition of WHSC1 restores antigen presentation. This occurs via an elegant epigenetic regulation of gene expression at the chromatin and DNA methylation level. In vivo studies in immunocompetent and immunocompromised mice also show an increased tumor homing of CD8⁺ T cells in mice treated with WHSC1 inhibitor, supporting the hypothesis that the antitumor effect following WHSC1 inhibition requires a fully functional immune system.

RESULTS

WHSC1 levels positively correlate with the presence of an immunosuppressive microenvironment in PCa:

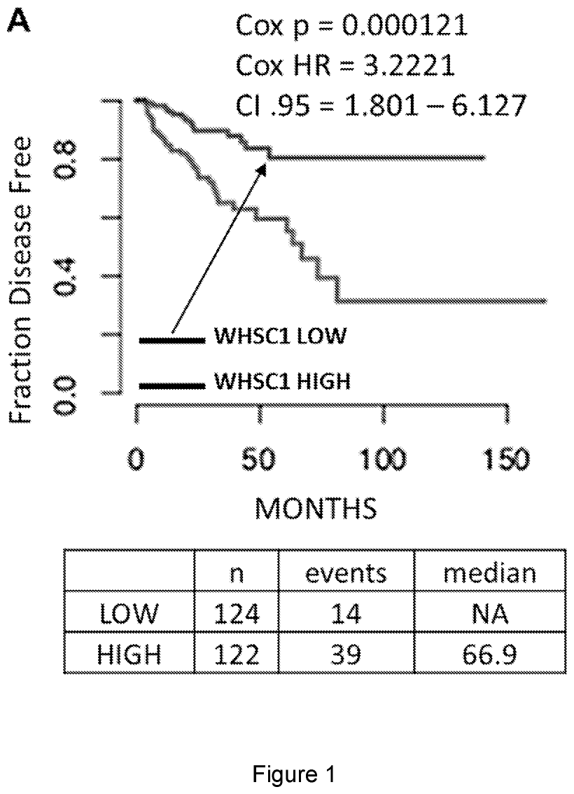

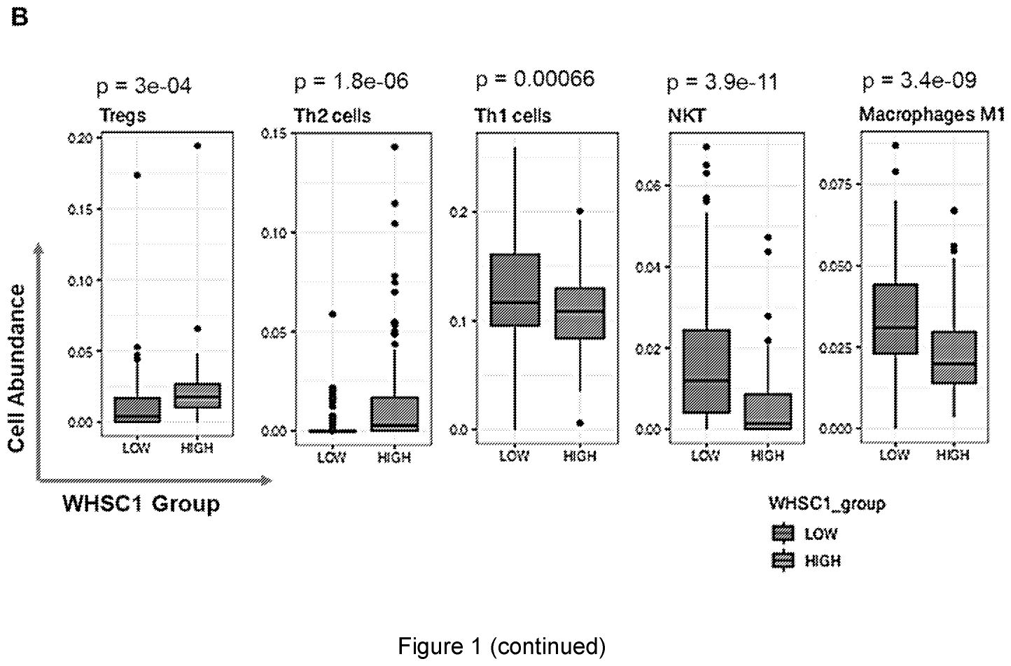

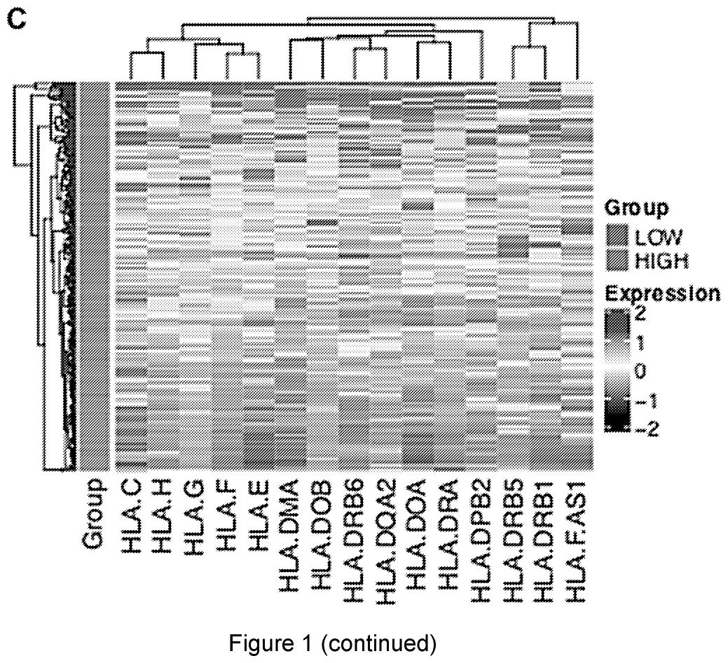

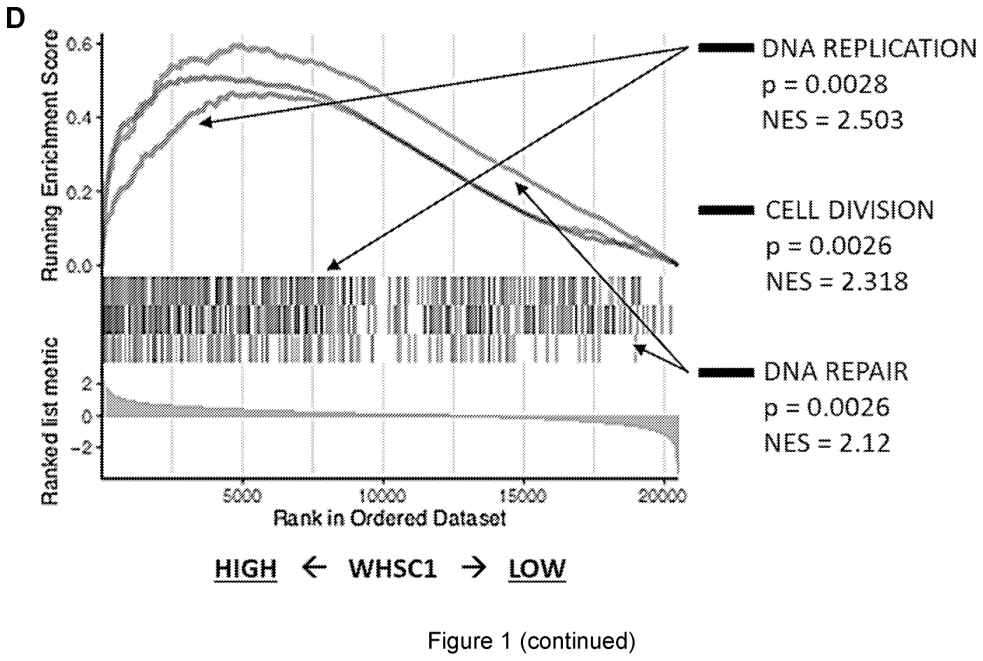

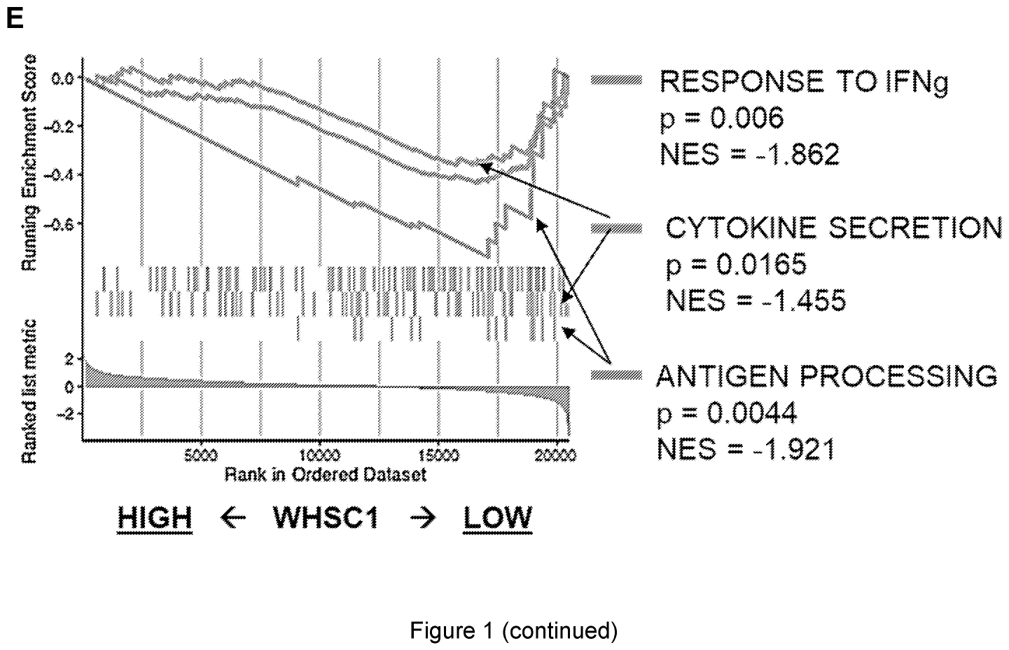

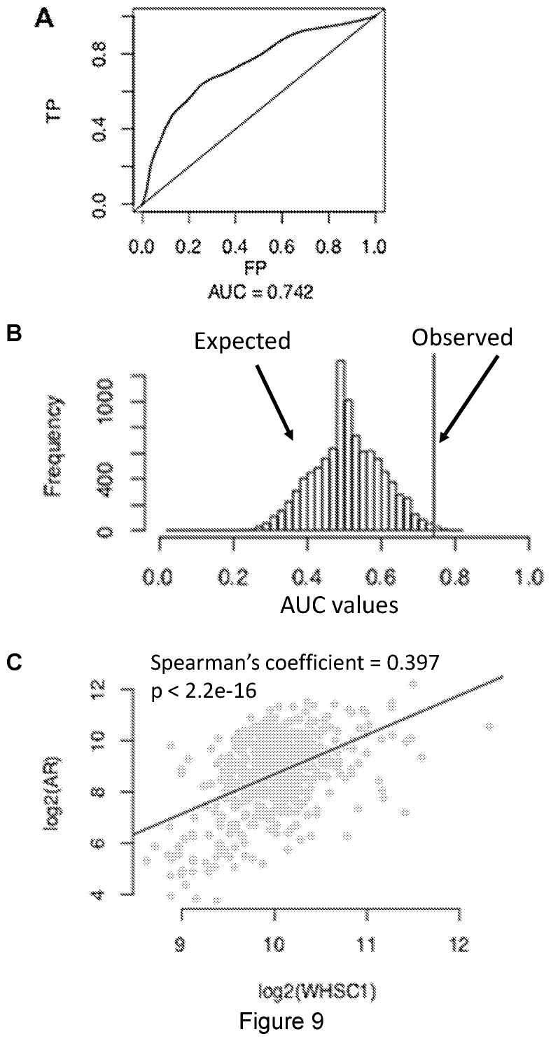

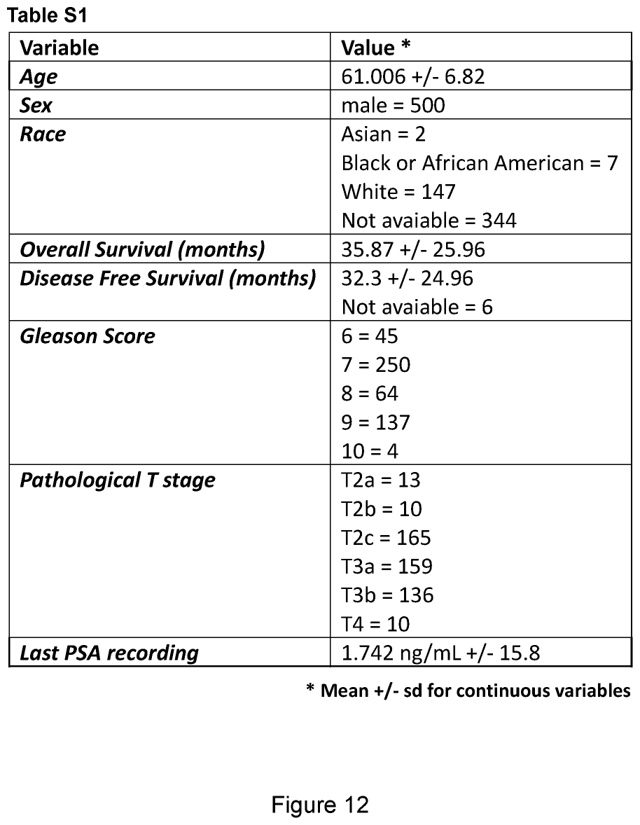

We first evaluated WHSC1 expression in 489 PCa samples from TCGA collected following radical prostatectomy (Table S1 (FIG. 12)) and its association with patients disease free survival (DFS). This analysis revealed that patients with elevated WHSC1 expression (top 25%) have significantly shorter DFS than patients with low WHSC1 levels (bottom 25%) (FIG. 1A) (Cox p=0.0001, Cox HR=3.2516, CI 0.95=1.763−5.996). Raising PSA concentration post therapy in PCa are often indicative of disease recurrence and we found that WHSC1 gene expression levels are a modest, but significant, predictor for biochemical recurrence with an AUC of 0.742 (FIG. 9A-B)(empirical p=0.0046). Moreover, PCa is an androgen-driven disease, and we found a positive correlation between WHSC1 and AR expression (FIG. 9C) (Spearman's coeff=0.4, p<1e-4). We then investigated whether WHSC1 expression levels correlate with the presence of specific immune populations and found that patients with elevated WHSC1 have a highly immunosuppressive tumor microenvironment, composed of high Th2 and T reg and low Th1 cells, NKT cells and M1 macrophages (FIG. 1B) (pv<0.001, Student's t-test). Next, we interrogated TCGA RNASeq data to evaluate whether different WHSC1 levels correlate with altered HLA expression in the tumor, which would undermine the tumor's ability to present tumor antigens to the immune system. Indeed, patients with elevated WHSC1 were found with consistently low expression of HLA class I and class II genes (FIG. 1C). We then investigated which transcriptional pathways were altered in patients with high vs. low expression levels of WHSC1. Gene set enrichment analysis (GSEA) revealed that patients with elevated WHSC1 have increased expression of genes involved with cell proliferation pathways such as cell division, DNA replication and DNA repair (FIG. 1D). However, we also identified a downregulation of pathways involved with response to IFNg, antigen processing and presentation and cytokine secretion (FIG. 1E), suggesting that high levels of WHSC1 negatively correlate with the status of both tumor resident immune pathways and the antigen processing and presentation machinery (APM).

Immune and APM genes are transcriptionally regulated by WHSC1 in PCa.

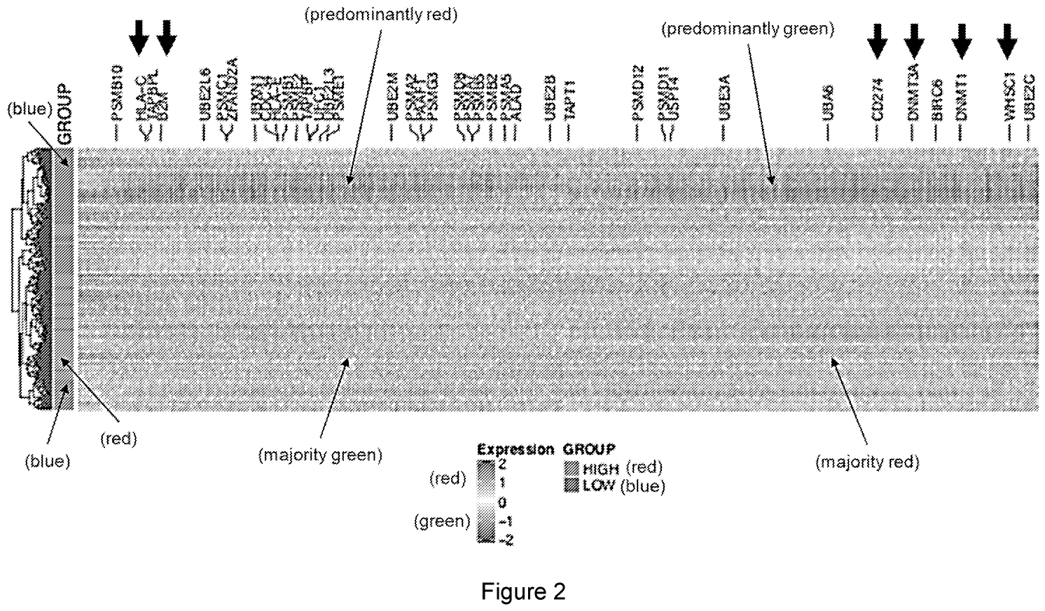

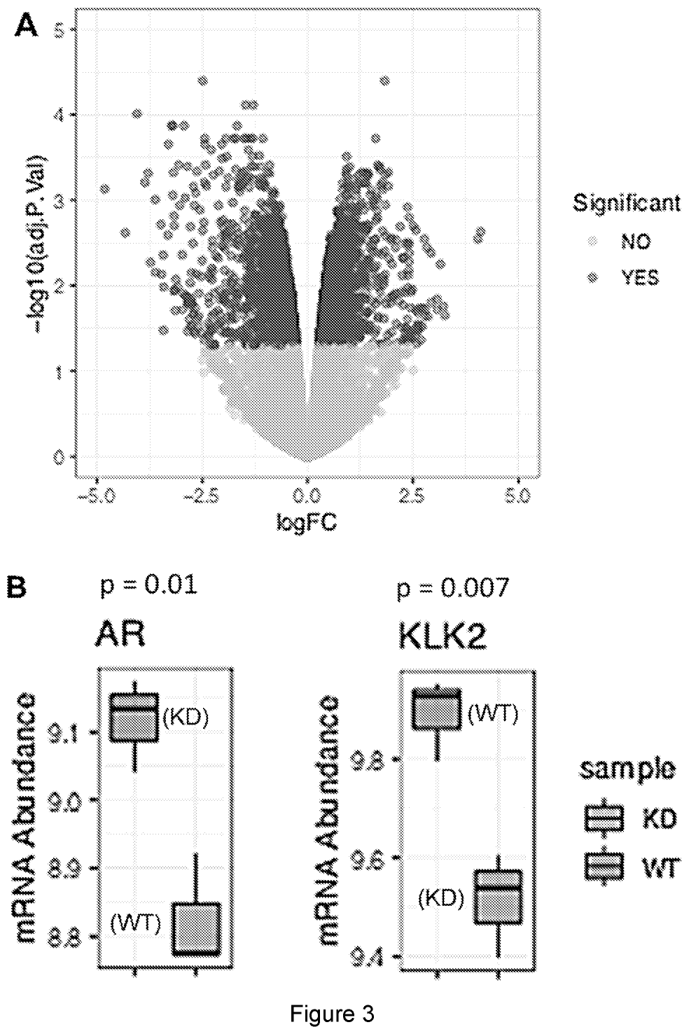

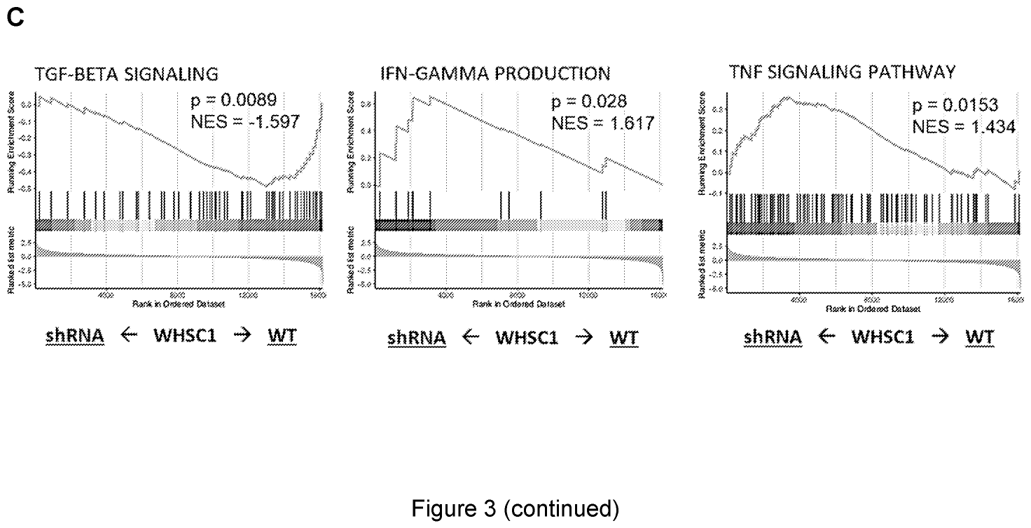

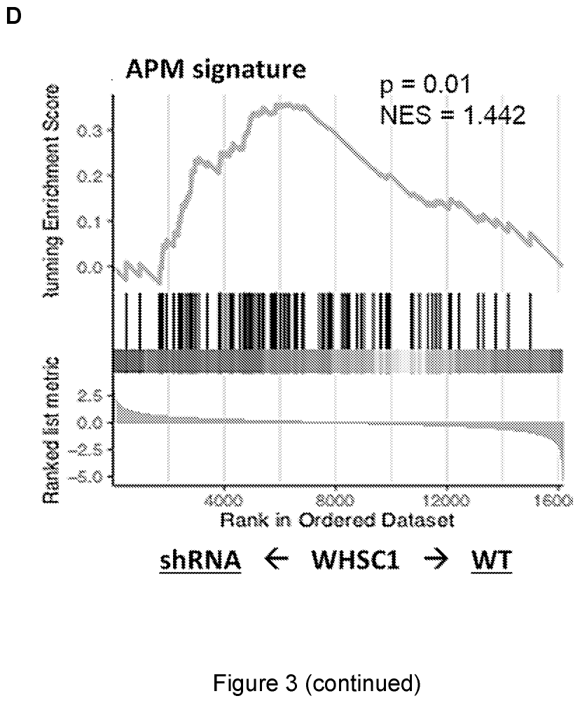

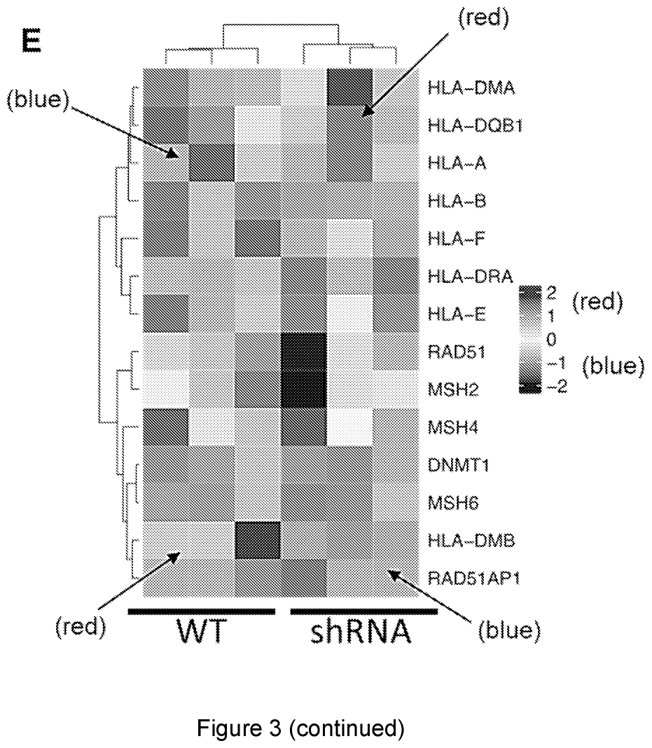

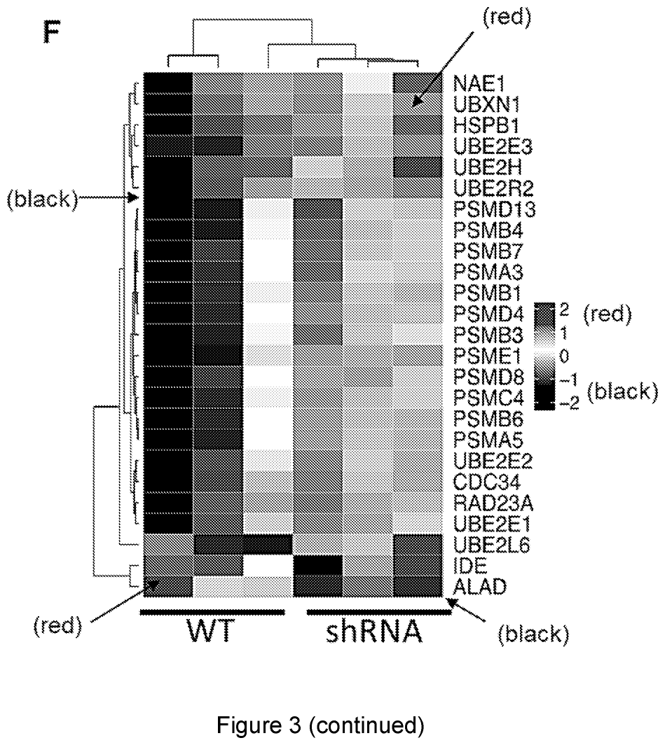

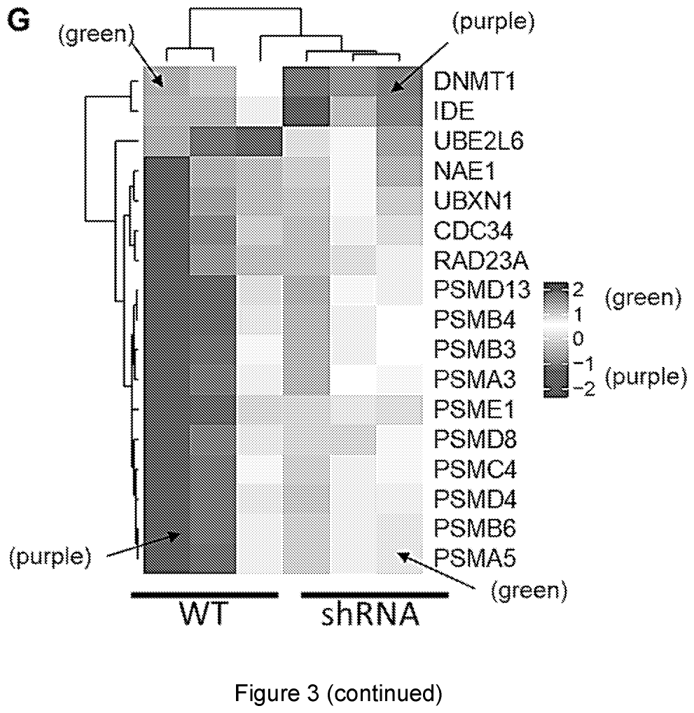

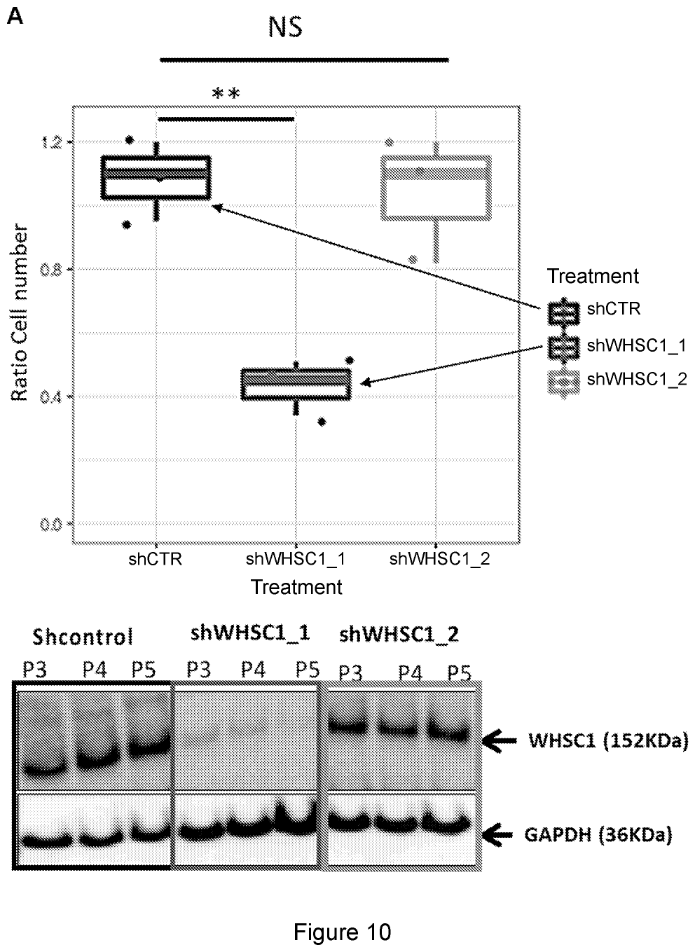

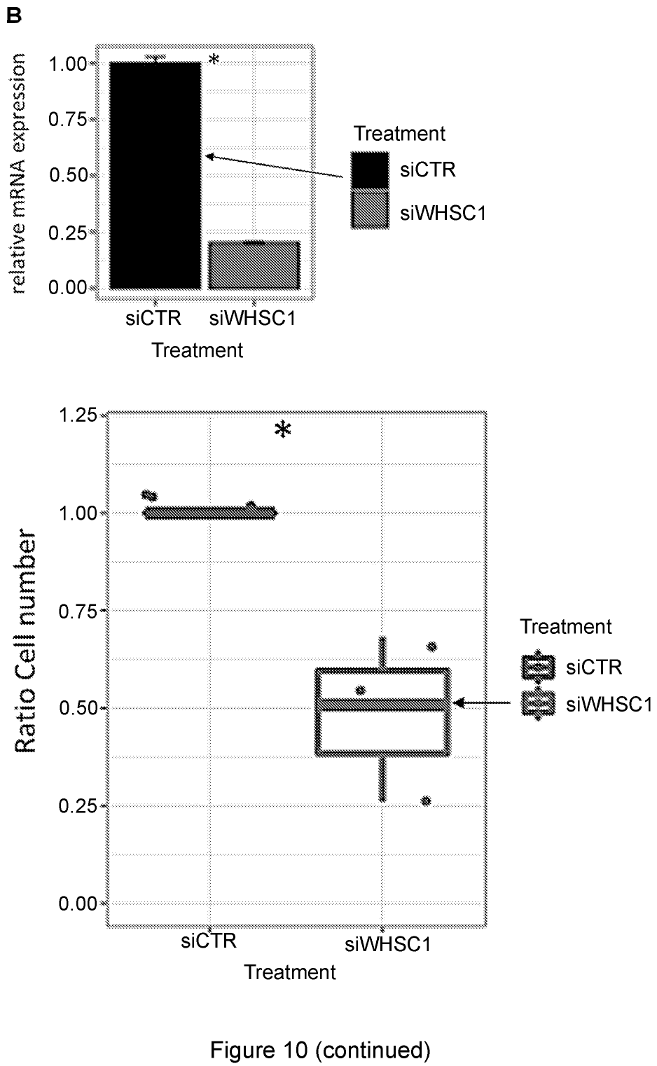

We sought to pinpoint potential mechanistic events that allow WHSC1 to regulate the expression of APM genes. To this end we downloaded publicly available ChIPSeq data from the Cistrome database and performed two independent analyses, one with H3K36me3 and one with H3K36me2 data. We first utilized H3K36me3 ChIPSeq data to predict WHSC1 targets exploiting the fact that WHSC1 can deposit both H3K36me2 and H3K36me3 marks, and that depletion of WHSC1 leads to loss of H3K36me3 regardless of the H3K36me2 mark. We generated a consensus list of H3K36me3 target genes intersecting data from three published ChIPSeq experiments in LNCaP cells (GSM1527830, GSM1527831, GSM1679107) and ran differential gene expression analysis for this signature in TCGA PCa tumors with high vs. low WHSC1. Results show that 36 APM genes, within the H3K36me3 signature, are differentially expressed (FDR<0.05). Moreover, DNMT1, DNMT3A and CD274 are all targeted by H3K36me3 and are all significantly upregulated in tumors with elevated WHSC1, while HLA-C and B2M are downregulated (FIG. 2). In order to validate our computational observations, we stably knocked down WHSC1 in C42 cells and noted a significant reduction in cell proliferation (FIG. 10A), also observed upon transient knockdown of WHSC1 in DU145 cells (FIG. 10B). Upon WHSC1 knock down, 3783 genes were differentially expressed (1834 down and 1949 up, FDR<0.05) (FIG. 3A) We first confirmed a downregulation of AR and its downstream target KLK2, indicating a transcriptional suppression of the androgen signalling (FIG. 3B). Gene set enrichment analysis (GSEA) revealed a downregulation of TGF-beta signaling and upregulation of IFN-gamma and TNF signaling (FIG. 3C). The APM pathway is also upregulated upon WHSC1 knockdown (FIG. 3D). When looking into the genes involved in the APM pathway we noticed an increase in HLA genes, parallel to a downregulation of DNMT1 (FIG. 3E), and a set of 25 proteosomal genes differentially expressed, with 23/25 being upregulated upon WHSC1 knockdown (FIG. 3F). Next, we utilized H3K36me2 ChIPSeq data from PC3 cells (GSM225904) to confirm previous results indicating APM gene being regulated by WHSC1 and residing in H3K36me2 sites. We intersected DEGs from our RNASeq data with the combined list of predicted targets of H3K26me2 and APM genes. Results show that most of the upregulated APM genes are within the H3K36me2 regions. DNMT1 was also within the H3K36me2 loci and downregulated upon WHSC1 knockdown (FIG. 3G). These results are consistent across different models (H3K36me3, LNCaP) and CRPC cell line (H3K36me2, PC3) and indicate a mechanistic and causative role for WHSC1 in regulating the expression of APM genes.

WHSC1 regulates protein degradation and immune components via DNA methylation

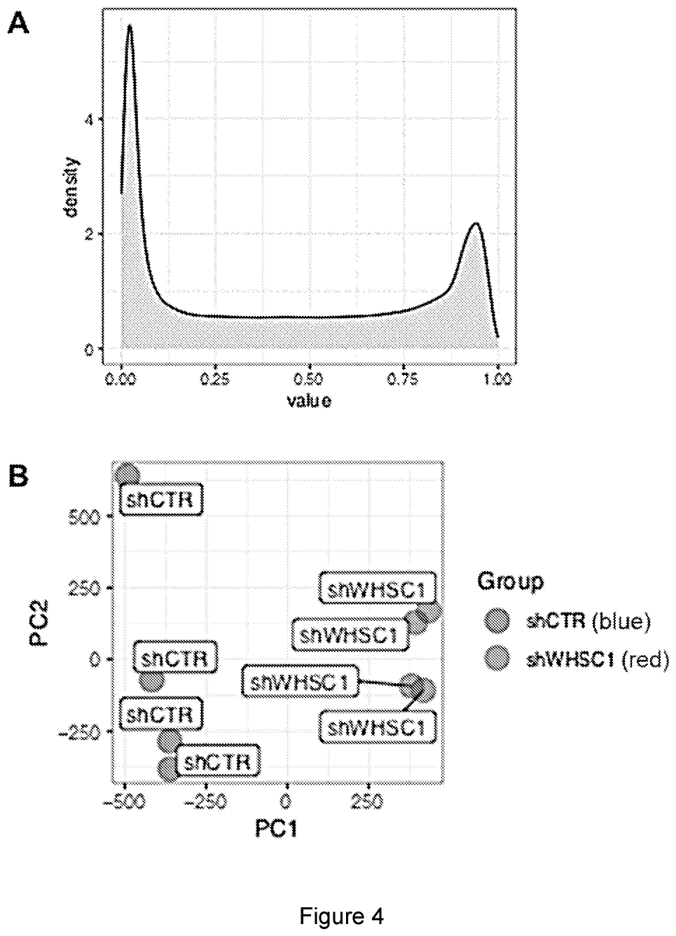

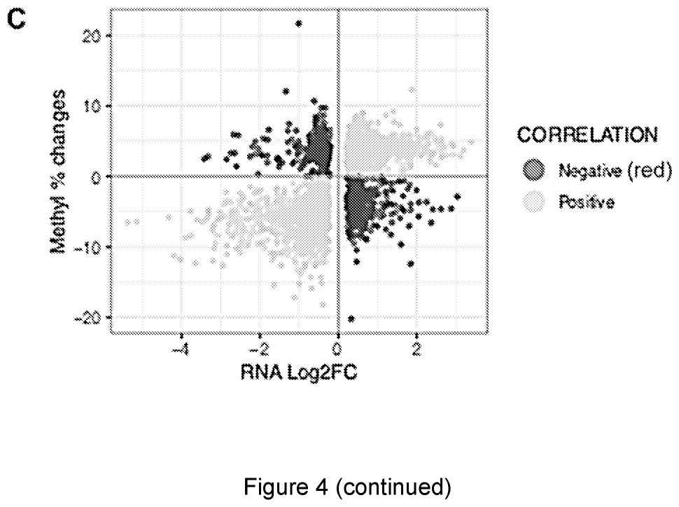

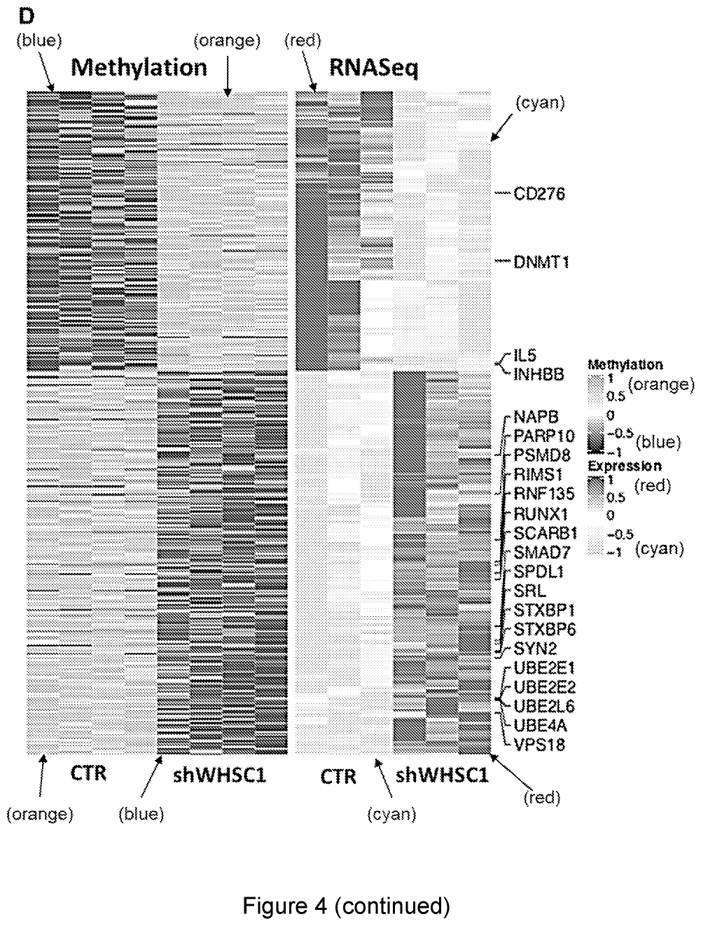

Our computational and transcriptional analyses indicate that WHSC1 regulates the expression of DNMT1, indicating a link between H3K26me2 and DNA methylation. To evaluate the link between DNA methylation and the expression of genes in immune pathways or APM, we performed methylomic analyses in C42 cells following WHSC1 knockdown (FIG. 4A-B).

A total of 2209 DEGs overlapped with genes containing differentially methylated probes and 651 of those negatively correlated with DNA methylation status upon WHSC1 knockdown (FIG. 4C-D). Within the genes that have reduced methylation and increased gene expression, we identified genes belonging to immune regulatory pathways and antigen processing. This includes six genes involved in peptide proteosomal degradation (UBE2E1, UBE2E6, UBE2L6, UBE4A, RNF135 and PSMD8); RUNX1, which is involved in promoting class I MHC expression (Howcroft et al., J Immunol. 2005; 174(4):2106-15. Epub Feb. 9, 2002 . doi: 10.4049/jimmuno1.174.4.2106. PubMed PMID: 15699141); SMAD7,which negatively regulates the immunosuppressive TGF-β signaling (reviewed in (Stolfi et al., Int J Mol Sci. 2013; 14(12):23774-90. Epub Dec. 10, 2013 . doi: 10.3390/ijms141223774. PubMed PMID: 24317436; PMCID: PMC3876077); a number of membrane trafficking proteins (SRL, RIMS1, STXBP6, NAPB); IL6R, increased during PCa development (Azevedo et al., World J Clin Oncol. 2011; 2(12):384-96. Epub Dec. 16, 2011 . doi: 10.5306/wjco.v2.i12.384. PubMed PMID: 22171281; PMCID: PMC3235657) and in breast cancer (Weng et a., Mol Cancer. 2019; 18(1):42. Epub Mar. 20, 2019. doi: 10.1186/s12943 0988-0. PubMed PMID: 30885232; PMCID: PMC6421700); INHBB, which belongs to the TGF-β family and is increased in PCa (Hofland et al., Endocrinology. 2012; 153(12):5726-34. Epub Feb. 10, 2012. doi: 10.1210/en.2011-2065. PubMed PMID: 23024260); IL5, which was shown to promote cancer metastases modulating the TME (Zaynagetdinov et al., Cancer Res. 2015; 75(8):1624-34. Epub Feb. 19, 2015. doi: 10.1158/0008-5472.CAN-14-2379. PubMed PMID: 25691457; PMCID: PMC4401663; Simson et al., J Immunol. 2007; 178(7):4222-9. Epub Mar. 21, 2007. doi: 10.4049/jimmuno1.178.7.4222. PubMed PMID: 17371978); PARP10, which limits cell proliferation and metastases (Zhao et al., Oncogene. 2018; 37(22):2921-35. Epub Mar. 9, 2018. doi: 10.1038/s41388-018-0168-5. PubMed PMID: 29515234); and CD276, which is elevated in several cancers and is a potential immunotherapeutic target (Seaman et al., Cancer Cell. 2017; 31(4):501-15 e8. Epub Apr. 12, 2017. doi: 10.1016/j.ccell.2017.03.005. PubMed PMID: 28399408; PMCID: PMC5458750). Lastly, we noticed that probes associated with the DNMT1 gene had increased methylation with a parallel reduction in DNMT1 gene expression (FIG. 4D). These results indicate that WHSC1 regulates tumor-resident immune pathways and APM via both modifying histones and the DNA methylation status.

WHSC1 epigenetically regulates genes in the APM by changing chromatin status

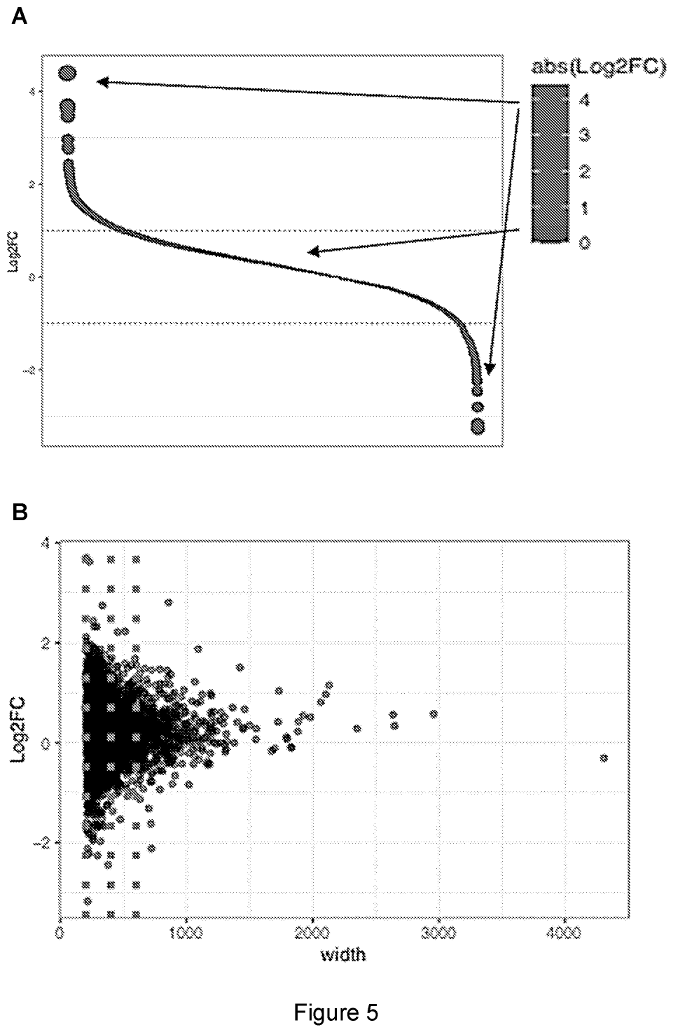

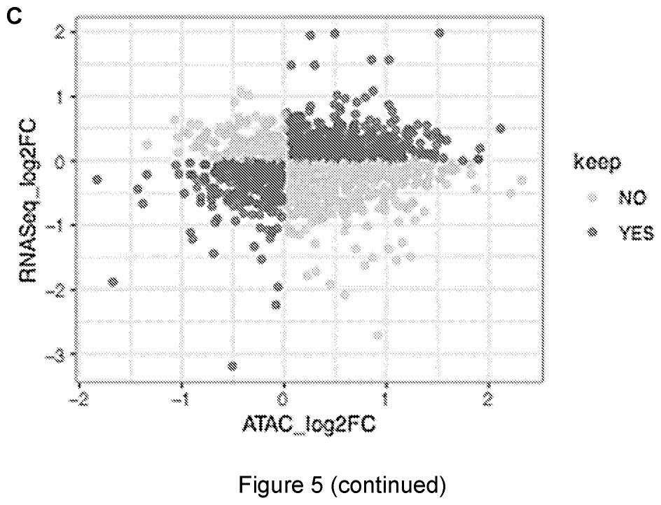

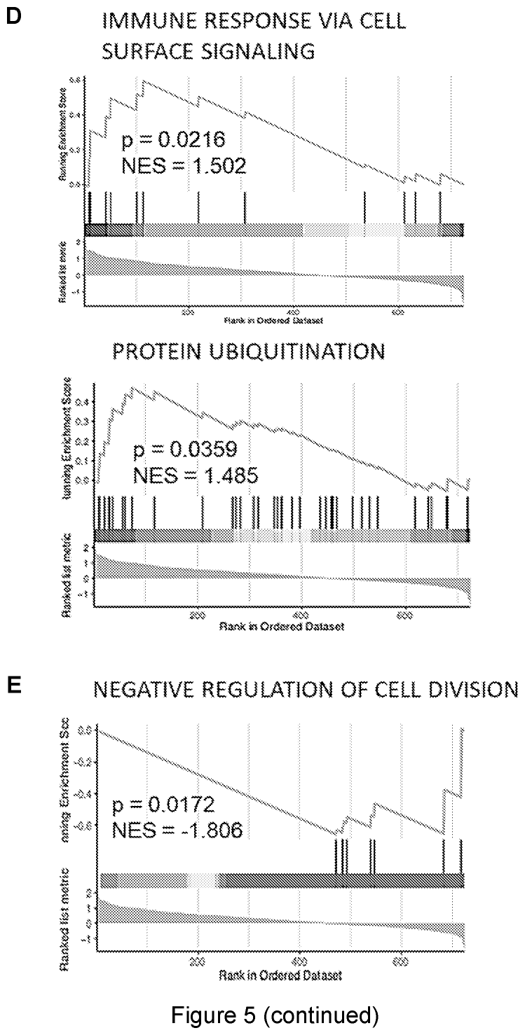

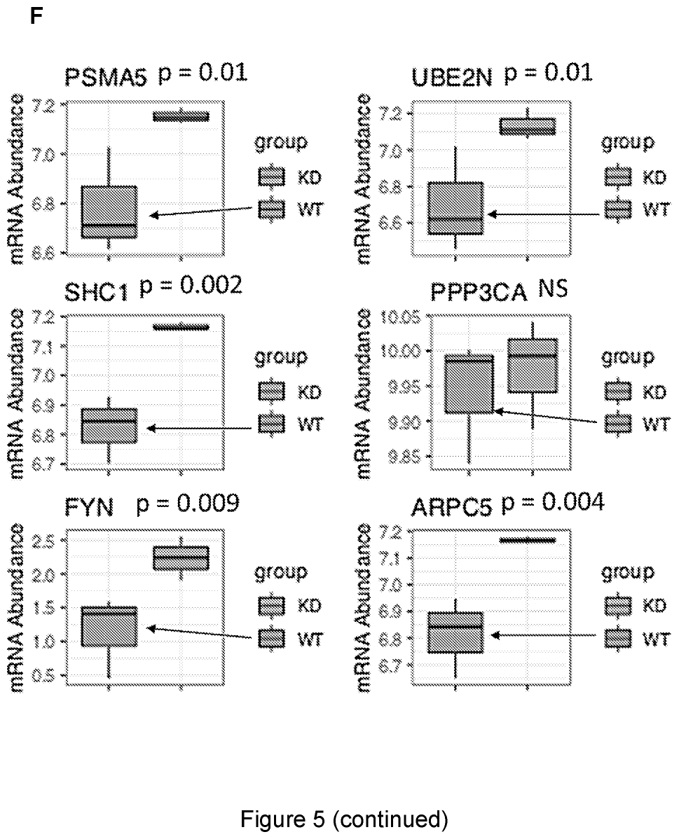

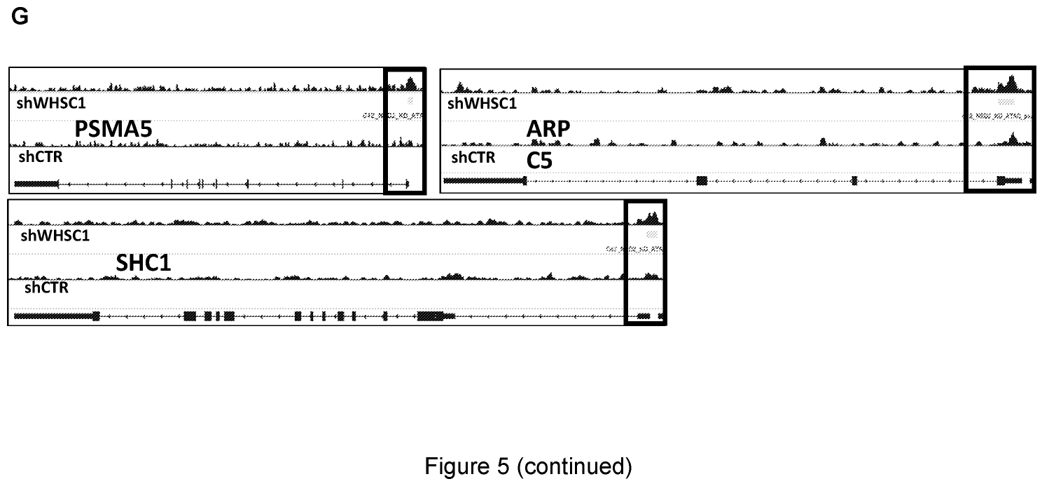

To further narrow the mechanistic role of WHSC1 in modulating APM genes, we performed ATACSeq analysis of C42 cells following knockdown of WHSC1. We first created a consensus peak list, compared the peak intensity between the two conditions and noticed that the biggest differences were, as expected, in the peaks for mono- or di-nucleosomal regions with no differences in larger peaks (FIG. 5A-B). After annotating genes to the peaks, we kept those with open chromatin and increased gene expression, or voce versa, as indicated by a positive correlation between the log fold changes in ATACSeq and RNASeq (FIG. 5C), indicating open chromatin and increased gene expression, or vice-versa, and performed GSEA analysis. Within the results, we identified an upregulation of genes in immune signaling and protein ubiquitination pathways (FIG. 5D) and downregulation of genes in proliferative pathways (FIG. 5E). Next, we evaluated the presence of peaks at the gene level and demonstrated that increased peak magnitude results in increased expression of the genes involved in the upregulated pathways (FIG. 5G).

These results further extend our observation that WHSC1 is a unique causative factor in modulating antigen processing and presentation in PCa via epigenetic regulation of gene expression.

WHSC1 and DNMT1 expression reflect tumor phenotype in-vivo:

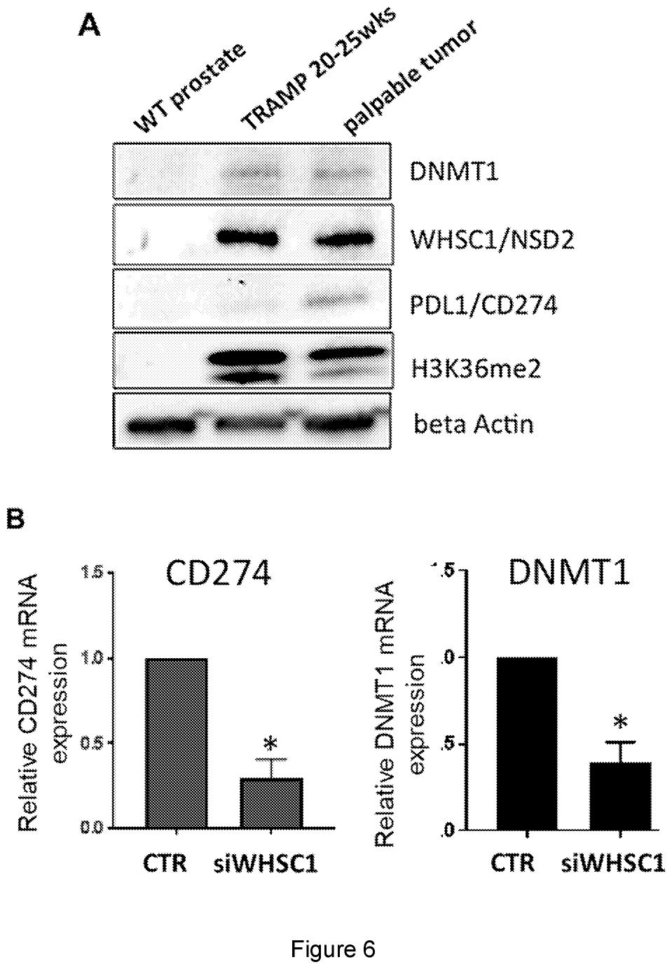

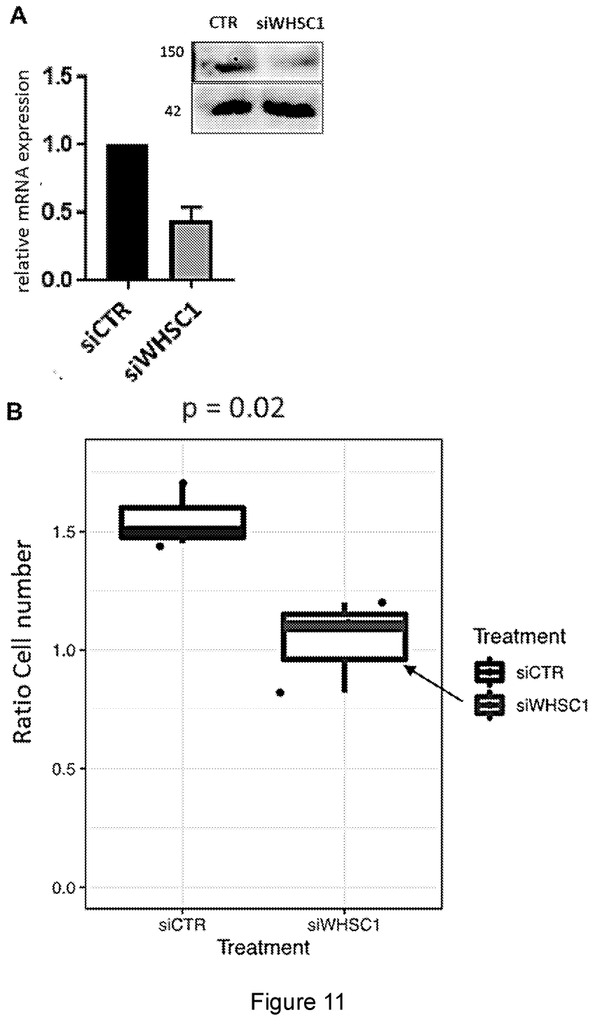

Following the in vitro experiments, we sought validate our findings in vivo. We used the TRAMP mouse as in vivo model of PCa to evaluate the expression of WHSC1, CD274/PD-L1, DNMT1 and the levels of H3K36me2 in prostates isolated from healthy WT mice, established tumors from the TRAMP mice, at 20/25 weeks of age and as aggressive palpable tumors (˜35 weeks). Healthy WT prostates have no detectable expression of any of the above proteins while higher levels of DNMT1, WHSC1, CD274/PD-L1 and H3K36me2 are detected in the tumor samples via western blot (FIG. 6A). We then tested whether the effect of WHSC1 knockdown in human C42 cells was reproducible in the murine TRAMP C2 cells, as proxy for potential in vivo effects. Knockdown of WHSC1 via siRNA led to a significant reduction in cell proliferation (FIG. 11A-B) and increased transcript levels of DNMT1 and CD274 (FIG. 6B).

Pharmacological inhibition of WHSC1 upregulates MHC molecules and increases immune infiltration in vivo:

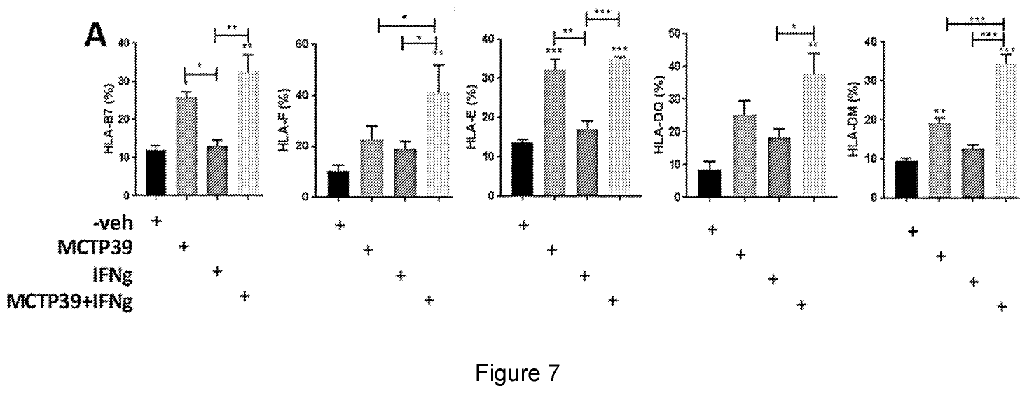

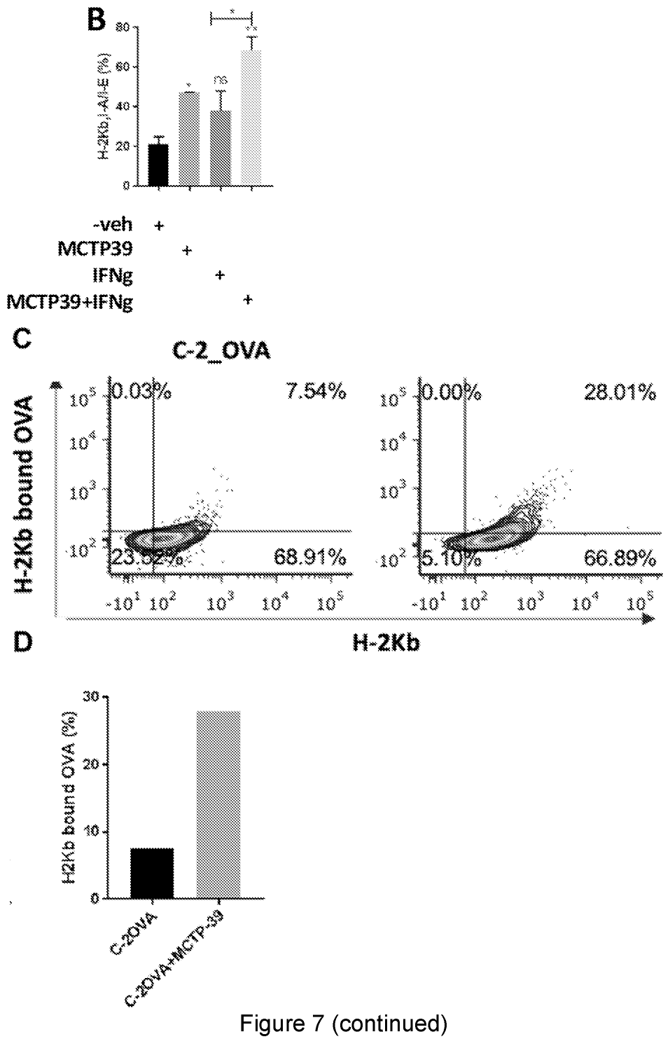

We investigated whether pharmacological inhibition of WHSC1 (with MCTP-39) can recapitulate the upregulation of MHC-I/II that we observed in the RNASeq data upon WHSC1 knock down. Human (C42) and murine (TRAMP C2) cells were treated with MCTP-39 and IFNg prior to flow cytometry analysis. The combined treatment had an additive effect on upregulating HLA-B7, HLA-F, HLA-E, HLA-DQ, HLA-DM (FIG. 7A) and murine H2Kb I-A/I-E (FIG. 7B). We then tested whether antigen-bound MHC was also elevated upon WHSC1 inhibition using OVA-overexpressing TRAMP C2 cells, and demonstrated that treatement with MCTP39 increased the OVA-bound H2Kb fraction (FIG. 7C-D).

A functional immune system is needed to mediate anti-WHSC1 tumor growth

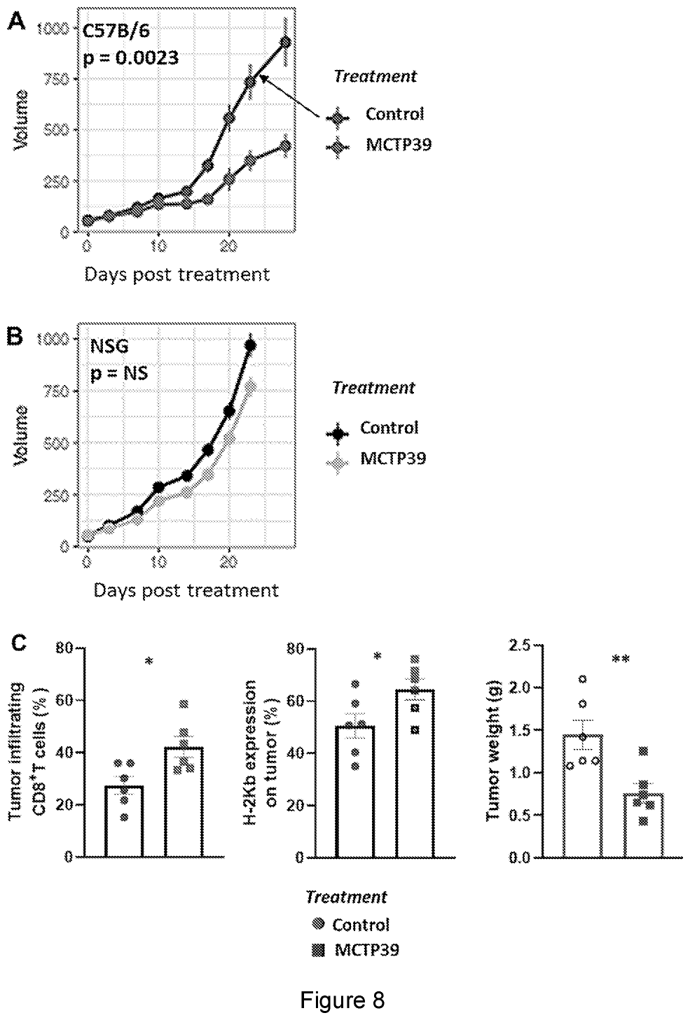

Hypothesizing that higher tumor antigen presentation would affect tumor growth and the levels of infiltrating immune cells in the tumor, we grafted C57B/6 mice with TRAMP C2 cells and administered MCTP39 (10 mg/kg) for four weeks IP and evaluated tumor size weekly and immune infiltration at endpoint. Tumor growth was significantly reduced upon treatment with MCTP39 (FIG. 8A) (p=0.0023), and this effect was not observed when we grafted the same exact number of TRAMP C2 cells in immunocompromised NSG mice (FIG. 8B). Moreover, the reduction in tumor growth in C57B/6 mice was accompanied by increased H2Kb expression, increased CD8⁺ T cells infiltration and reduced tumor weight (FIG. 8C).

These results indicate that that the anti-tumor effect observed following WHSC1 inhibition requires the presence of a functional immune system for optimal tumor control.

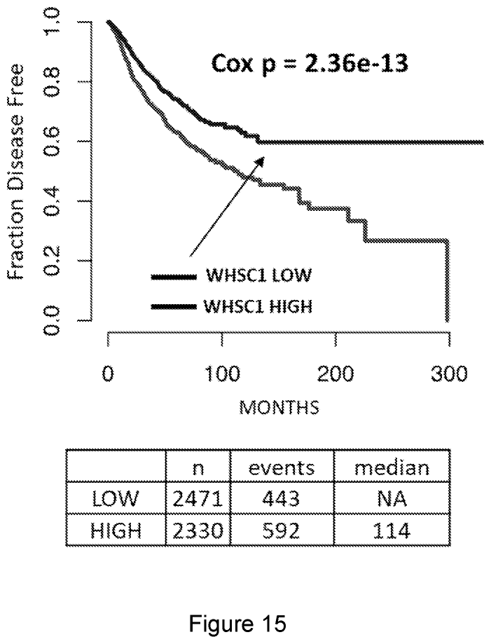

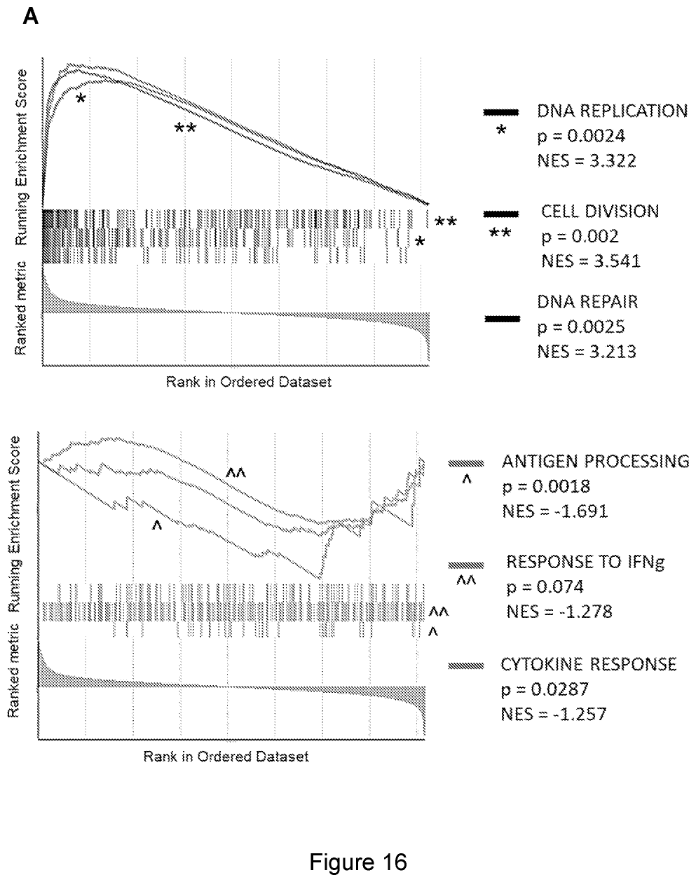

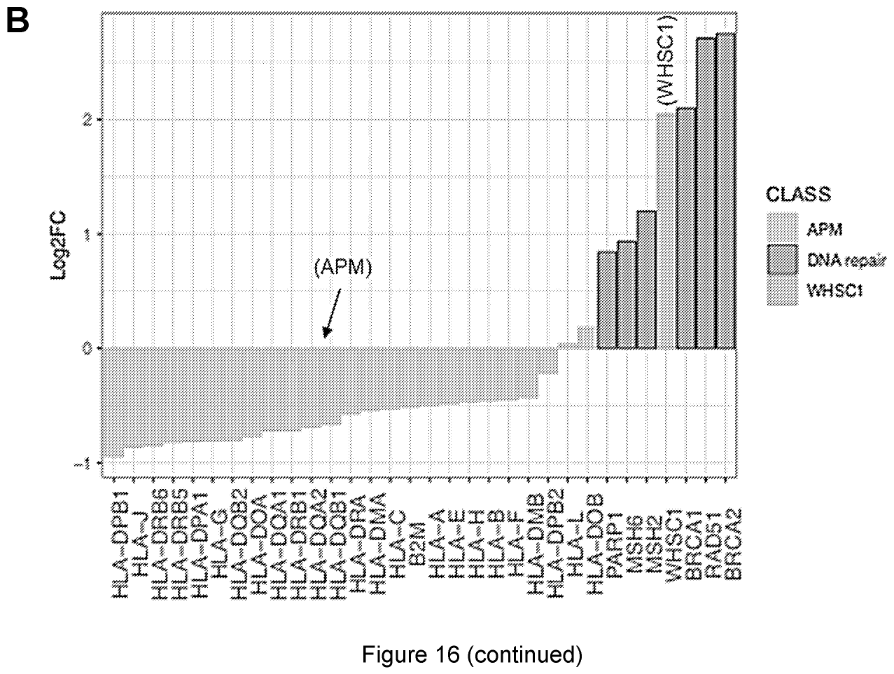

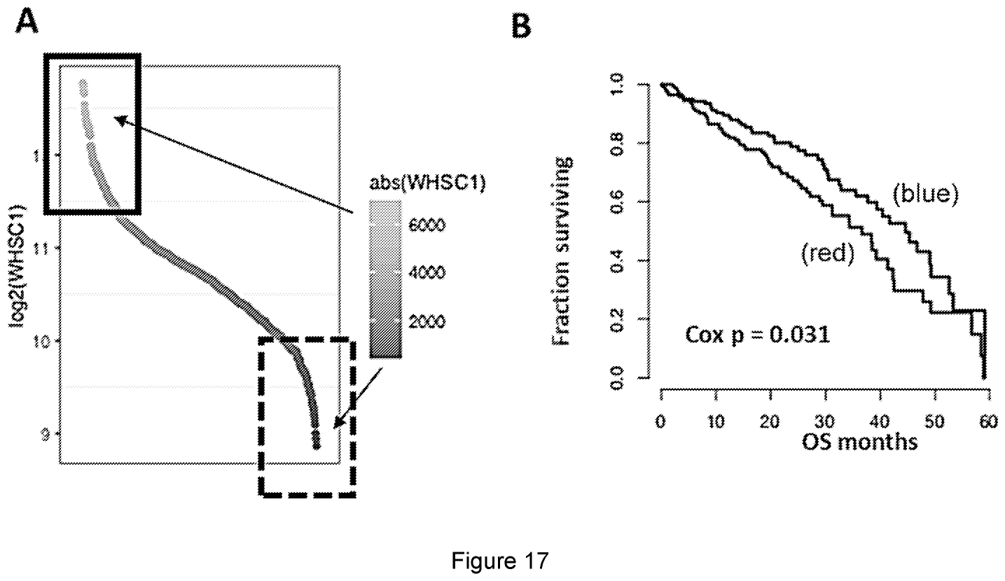

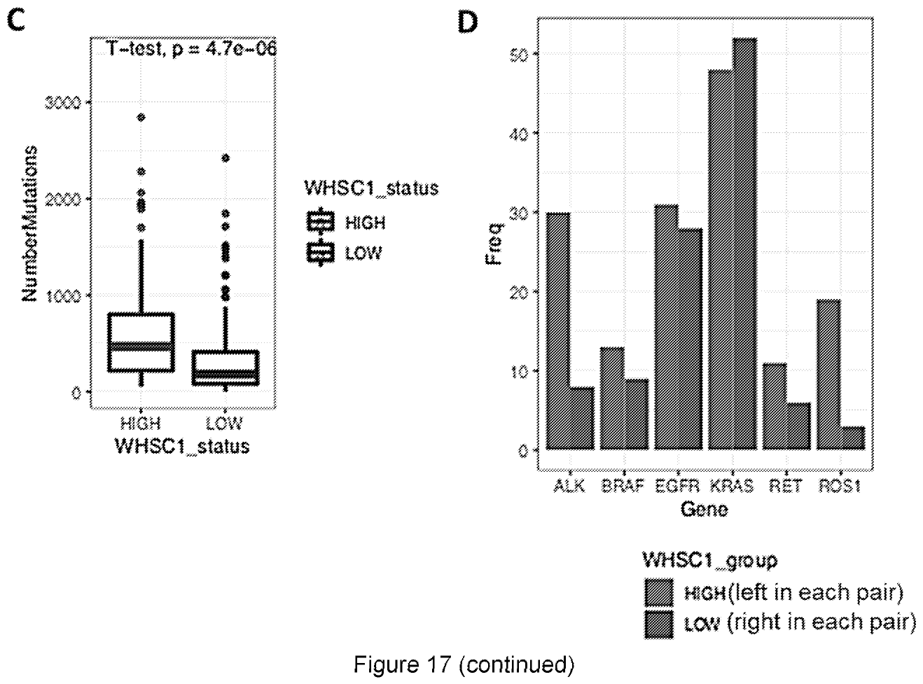

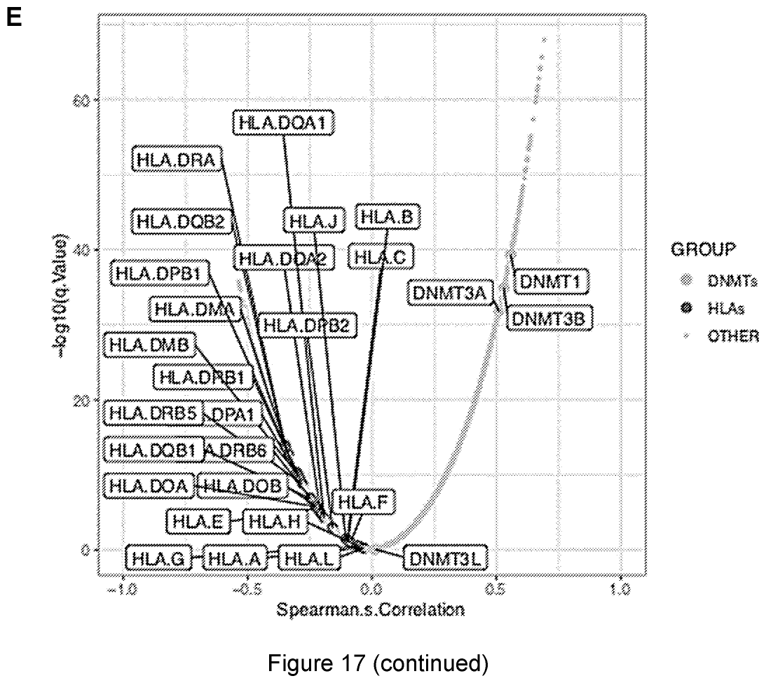

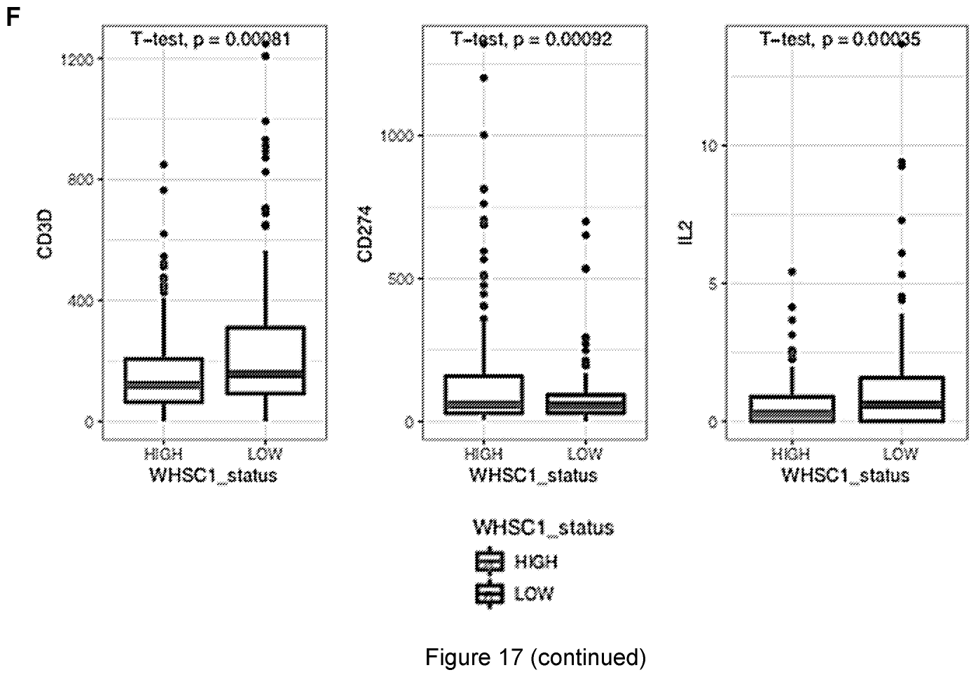

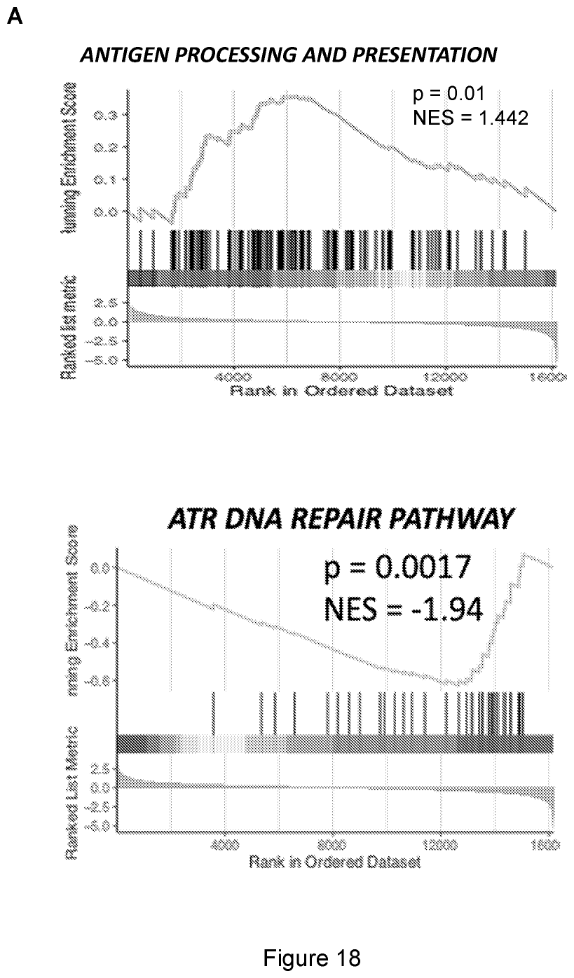

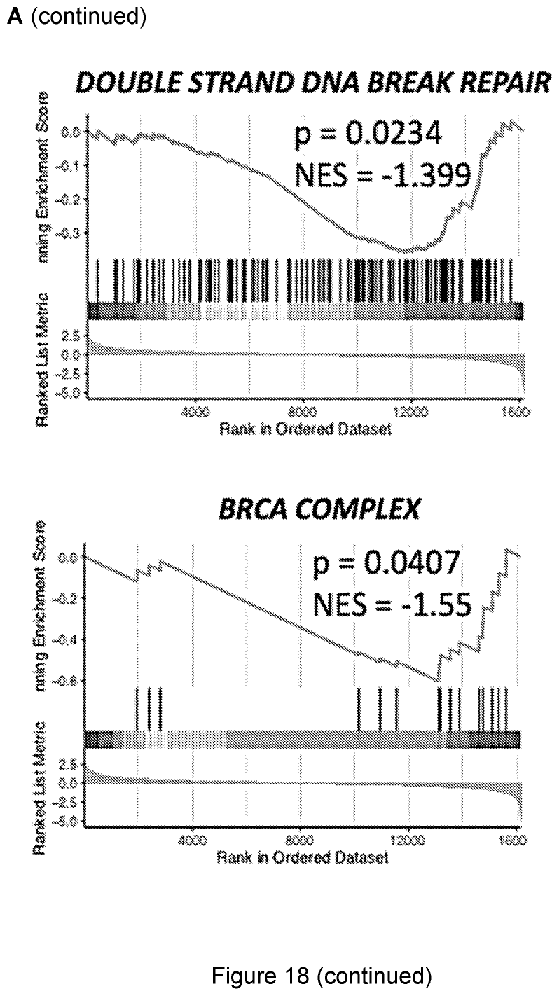

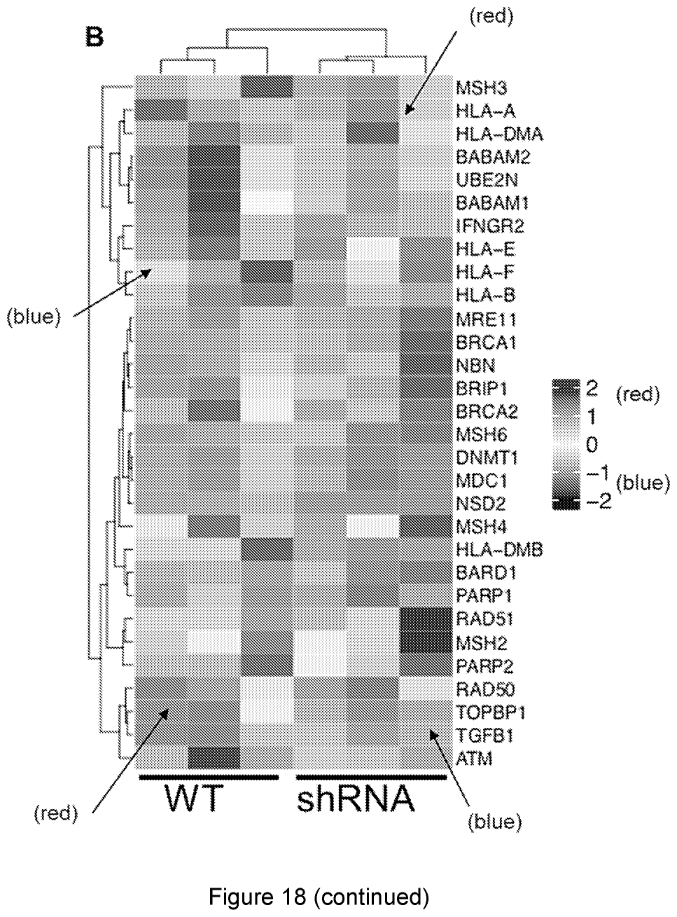

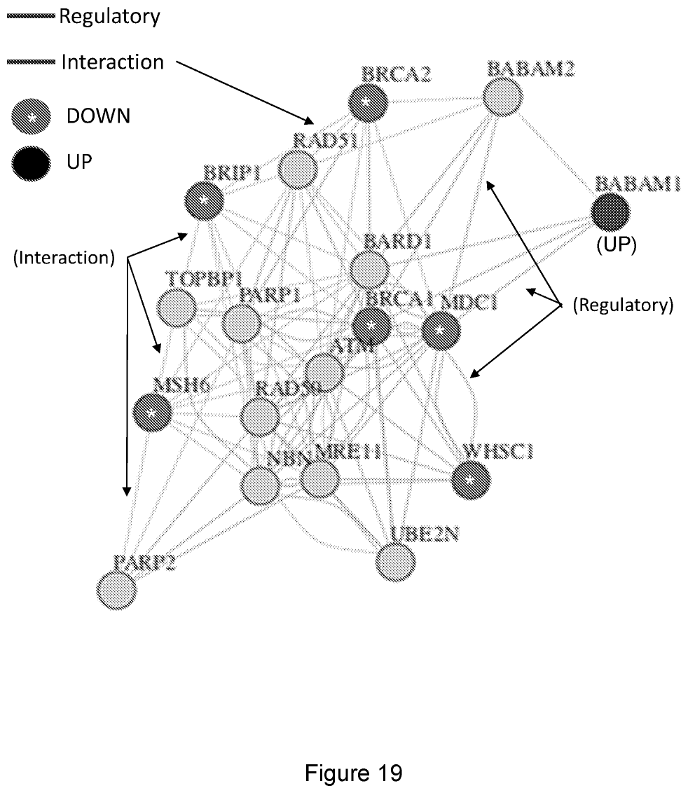

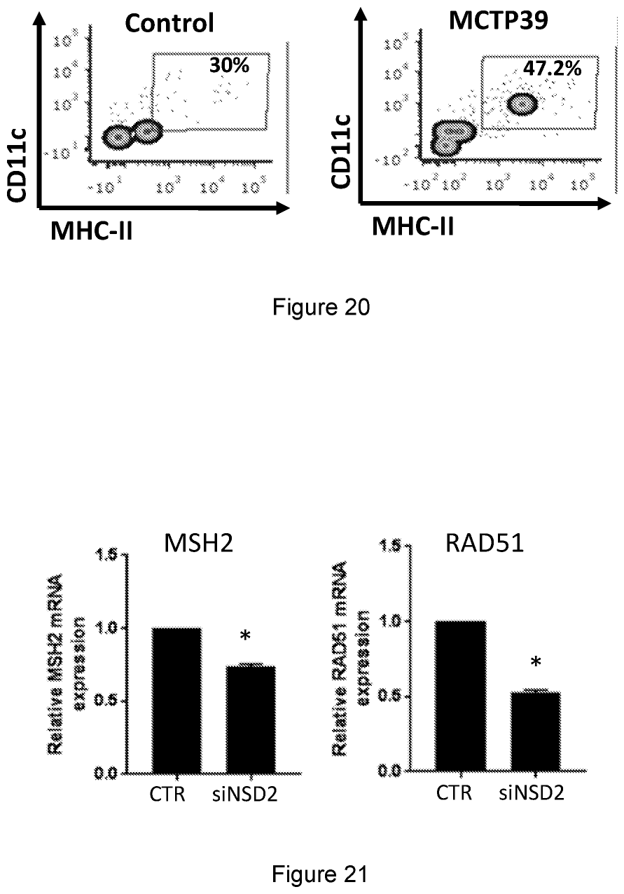

Additional data from these and related studies is provides in FIGS. 15-21. Data from lung cancer patients is provided in FIG. 17A-F.

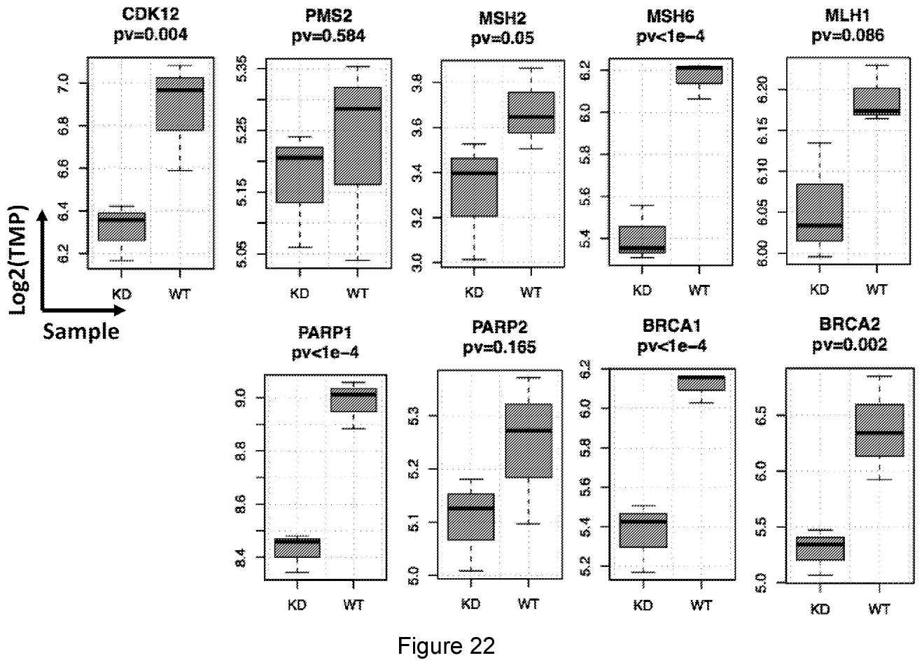

PCa and anti-PD1 therapy: Because of the suggested role of WHSC1 in regulating the expression of APM and HLA genes and in promoting the infiltration of PD1⁺ T cell in the prostate, we sought to identify other indicators that WHSC1 inhibition might increase the anti-tumor effect of anti-PD1 therapy. We parsed our RNASeq data to evaluate changes in genes involved with anti-PD1 response. Results show that WHSC1 knock down significantly downregulates CDK12, MSI (Microsatellite Instability genes) (MSH2, MSH6 and MLH1), PARP1/2 and BRCA1/2 (FIG. 22). These results suggest that WHSC1 inhibition can define a transcriptional signature that can synergize with anti-PD1 therapy to maximize anti-tumor immune response in PCa.

METHODS

Cell lines: TRAMP C2 cells were a kind gift from Dr. Barbara Foster, they were maintained in DMEM with 10% FBS supplemented with 1 nM DHT, 0.008 mg/ml insulin and penstrep. C42 cells were maintained in RPMI1640 with 10% FBS and P/S. C42 cells were validated via microsatellite PCR at the Roswell Genomics core. Both cell lines were mycoplasma negative.

WHSC1 knockdown: shRNA knockdown of WHSC1 in C42 cells was prepared by the Roswell Park Gene Editing shared resource. Transient knockdown experiments in TRAMP C2 cells were made using SiWHSC1 (4390771, ThermoFischer Scientific) or SiCTR (4390843, ThermoFischer Scientific), and those in DU145 cells using siWHSC1 (SR305101) or siCTR (SR30004) from Origene.

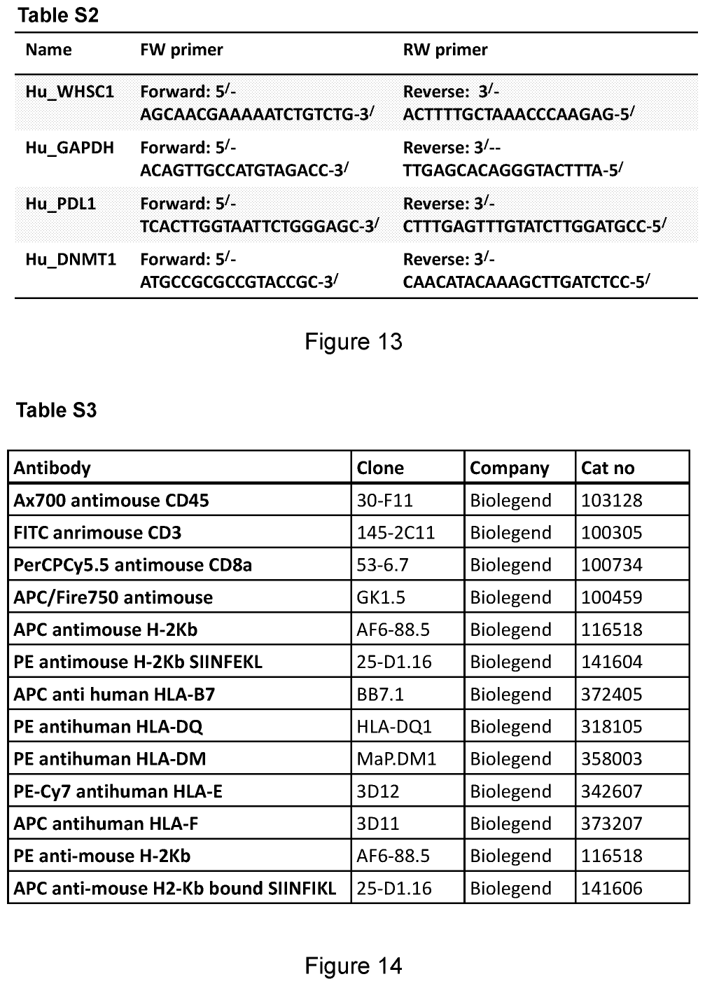

For siRNA knockdown, 200×10³ C4-2 cells or TRAMP C-2 cells in 200 μl optiMEM were plated in 24 well plate for overnight and next day were transfected with or without siWHSC1 (Invitrogen) using lipofectamine RNAimax at 37 C, 5%CO2. After 6 hours media was gently aspirated and replenished with fresh RPMI1640 or DMEM supplemented with DHT and further incubated at 37C, 5%CO2 for 48 hours. Cells were harvested after 48 hours for RNA isolation using TRIzol (Invitrogen, 15596026) for qPCR or for protein isolation by RIPA buffer for western blot. The concentration of RNA was measured by nanospectrometer and for RT-qPCR, 2 ng/μl of RNA were used for cDNA synthesis using iscript cDNA synthesis kit (Biorad, 170-8891). SYBER Green/Rox qPCR master mix (ThermoFischer Scientific, K0221) was used to analyze the expression of WHSC1, PDL-1, DNMT1 and GAPDH. The primer sequence is for these genes is provided (Table S2 (FIG. 13).

OVA overexpression: Soluble ovalbumin (OVA) gene, which was amplified from pCI-neo-sOVA (Addgene plasmid 25098), and monomeric enhanced green fluorescent protein (mEGFP) gene, which was amplified from mEGFP-N1 (Addgene plasmid 54767), were genetically fused via P2A translational skipping sequence and cloned in the Sleeping Beauty transposon plasmid with the human elongation factor 1α promoter. This plasmid, pT2-EF-OVA-mEGFP, was electroporated together with the Sleeping Beauty Transposase plasmid, pCMV(CAT)T7-SB100 (a gift from Zsuzsanna Izsvak; Addgene plasmid #34879), into TRAMP-C2 by Nucleofector 4D instrument. The electroporated cells were kept in maintenance medium (DMEM supplemented with 10% FBS, 0.005 mg/ml bovine insulin, 1 nM DHT and cell sorted based on mGFP expression using FACS Aria I cell sorter. The expression of OVA on sorted cells were confirmed by western blotting using rabbit polyclonal OVA antibody (ab186717) at 1:4000 dilution and flow cytometry using PE anti-mouse H-2K^{b} bound to SIINFEKL (BioLegend) before incubating with or without MCTP-39 for 48 hours. The expression of OVA was analyzed after 48 hours by flow cytometry using BDLSRIIA cytometer and data was analyzed by FCS express 7 Research Edition.

Western Blotting: Protein concentration was measured using BCA kit and 30 μg of protein was loaded into SDS-PAGE gel from either transfected or untransfected C4-2 or TRAMP C-2 cells. The protein from gel was transferred into PVDF and further incubated with human (anti-WHSC1, Abcam, ab225625) or mouse (anti-WHSC1, Abcam, ab75359) primary antibody to WHSC1 using 1:2000 dilution for C4-2 lysate and 1:1000 for TRAMP C-2 protein lysate. The primary antibody was further detected using either goat anti-rabbit (Abcam, dilution 1:10,000) or goat anti-mouse (Abcam, dilution 1:5000) while the house keeping gene, GAPDH in both C4-2 and TRAMP C-2 protein lysate was detected using anti-GAPDH antibody at 1:50,000 dilution (Abcam, ab181602).

Cell proliferation: Knock down of WHSC1 in C4-2 and TRAMP C-2 cells is discussed in previous sections. Cells were counted at 48, 96 and 144 hours. For pharmacological inhibition, cells either C4-2 or TRAMP C2 were seeded overnight at 4×10³ cells per 100 μl media in a 96 well plate, the following day cells were treated with vehicle control or different concentrations of MCTP-39 (0-10 μM) for 48 hours. After 48 hours, C4-2 or TRAMP C-2 cells were either counted or used for staining with MHCI/II antibodies. Briefly cells were detached using trypsin and washed with FACS buffer for 5 minutes at 300 g. The pellet was resuspended in FACS buffer and stained with MHC-I/II antibodies for 20 minutes at 4 C. After incubation, cells were washed with FACS buffer 2× and resuspended in 200 μl of FACS buffer before acquiring the data on a flow cytometer.

Mice and in vivo experiments: Male C57B/6J mice (6-8 weeks of age) and NSG mice (10-12 weeks of age) were obtained from the Roswell Park's Center For Immunotherapy breeding colonies. All in vivo experiments were made following institutional and IACUC regulations. In vivo experiments were not blinded. C2 cells (1×10⁶ cells/100 μl) were injected subcutaneously into the right flank of male C57BL/6J mice (n=6/group) or NSG mice (n=5/group) using a 27 G needle. Sample size was chosen based on pilot studies and chosen to include the potential loss of mice prior treatment. Tumor volume was monitored weekly with an electronic caliper and calculated as V=(W²×L)/2, where Vis tumor volume, W is tumor width and L is tumor length. When tumors reached 100 mm³, mice were randomized prior treatment with either MCTP-39 (10 mg/kg 5×/week/4 weeks) or vehicle control. Mice were euthanized either when tumors reached 2000 mm in any dimension, as per Institutional IACUC regulations, when mice show signs of advanced disease or after 4 weeks of treatment. Tumors were harvested and weighted, single cells suspension was prepared using the Tumor dissociation kit (Miltenyi Biotech) as per manufacturing instructions prior flow cytometry analysis.

Flow cytometry and antibodies: Single cell suspension from tumors was stained the antibodies listed in Table S3 (FIG. 14) for 20 min at 4° C. After staining, cells were washed and fixed with fixation buffer for 15 min at 4° C. followed by washing 2× with FACS buffer. Cells were resuspended in 200 μl of FACS buffer before acquiring data on BDLSR IIA flow cytometer. Data were analyzed using FCS express 7 Research Edition.

Analysis of published datasets: Publicly available prostate cancer datasets were retrieved from cBioportal (https://www.cbioportal.org/). Detailed statistical methods are explained below in the appropriate section. GSEA analysis on TCGA data was done using the package DOSE, clusterprofiler and enrichplot (github.com/GuangchuangYu/enrichplot) using gene signature downloaded from the GSEA website (gsea-msigdb.org/gsea/index.jsp). ChIPSeq data were downloaded from Cistrome (cistrome.org/). LNCaP cells ChIPSeq: GSM1527830, GSM1527831, GSM1679107. Peaks (annotated) present in at least two datasets, larger than 150 bp and scored at the top 10% were retained, for a total of 6512 unique genes. PC3 cells ChIPSeq: GSM225904. Precomputed TCGA immune infiltration data were downloaded from xCell.

RNASeq: RNA extraction and library preparation were performed by the Roswell Park Genomics Shared Resources. RNA libraries were constructed using the KAPA mRNA HyperPrep Kit (Roche Sequencing Solutions) and the libraries were sequenced on the Illumina NextSeq 500 sequencer with 2×75 cycle sequencing. Raw reads were compiled into fastq files, mapped onto the human hg38 reference genome using STAR and quantified at the gene level using the tximport R package. Genes differentially expressed between conditions were identified using limma. GSEA analysis was performed as described above. APM signature was generated using HLA genes and genes involved in the antigen processing and presentation.

ATACSeq: The ATACSeq libraries were sequenced using NextSeq 500 sequencer at 2×75 cycle sequencing. Raw data were processed with MACS2 and further processed using ChIPSeeker to annotate identify the genes within the genomic regions within ATAC peaks. To calculate the fold changes between the two conditions, we created a consensus list of genomic regions covered by both conditions (shCTR and shWHSC1) using the soGGi R Bioconductor package. Reads spanning over these regions were then quantified using Rsubread and used to calculate the log2FC. Results were then merged with RNASeq DEGs to identify those regions that positively correlate with RNASeq data, hence higher ATAC signal, higher gene expression. Those genes were then used for GSEA analysis ranking them based on the ATACSeq log2FC as described above.

Methylation analysis: Methylation analysis was performed using Illumina Infinium MethylationEPIC BeadChip Kit (Illumina Inc.). Raw files were processed using the Champ Bioconductor R package using default parameters. Methylation probes that coincided with known SNPs were removed. Probe IDs from the differentially methylated probe (DMP) list were merged with RNASeq DEG data, aggregated using mean intensity values at the gene level and correlated with RNASeq log2FC results to identify genes with reduced methylation and increased gene expression, or vice versa, upon WHSC1 knockdown.

Statistical methods: Survival analysis was performed dividing patients based on the upper/lower 25% of the expression levels of WHSC1, significance, HR and confidence intervals were calculated using cox proportional hazard model available in the R package survival. The AUC analysis to evaluate WHSC1 as predictor for biochemical recurrence was done using the R package survivalROC censoring for biochemical recurrence. Since TCGA does not offer the history of PSA testing per each patient over time, we used a threshold of PSA>0.4 ng/mL as described in. Significance for the AUC analysis was calculated by simulating 10,000 AUCs using randomly selected genes in the RNASeq dataset. The empirical p value was calculated by dividing the number of expected/simulated AUCs higher than our observed value by 10,000 (number of simulations). Significance when comparing two groups was calculated via two-tailed Student's t.test at a significance threshold of 0.05. When more than two groups were compared, one-way ANOVA with Tukey's post-hoc correction was used at significance threshold of 0.05. In both cases, barplots indicate the mean and standard error of at least three biological replicates unless specified otherwise. Growth curves in mice were compared using permutation test with 10,000 simulations via the statmod R package using the compareGrowthCurves function.

Discussion

The use of immunotherapy in prostate cancer has been pioneered by the use of vaccines targeting the prostate specific antigen (PSA) (Sipuleucel T/Provenge) or the prostate acid phosphatases (PAP) (Prostvac VF) that showed significant clinical benefits for patients with metastatic prostate cancer. However, there is still an incomplete understanding of the regulatory interface that mediates T cells homing into the prostate. The relevance of filling this gap in knowledge is highlighted by negative results or minimal benefits recorded in subsequent clinical trials testing immunotherapeutic approaches in patients with prostate cancer. Therefore, a thorough understanding of the mechanism by which PCa evades the immune system and limits T cell infiltration could have significant translational consequences.

Here we presented an epigenetic tumor-driven mechanism by which prostate tumors remain relatively cold due to increased levels of the epigenetic enzyme WHSC1. We provide data for its role in promoting immune evasion by rendering tumor cells less visible to the immune system. While not intending to be bound by any particular theory, it is considered that this is achieved by an elegant and coordinated downregulation of MHC molecules and APM genes through alteration of the chromatin status and by modifying the methylation of APM genes.

We first used bioinformatics approaches to investigate the relationship between the transcript levels of WHSC1 and genes in immune-related pathways, and presented correlative and transcriptional data demonstrating that increased WHSC1 transcripts negatively correlate with HLA levels and with the presence of an immuno-permissive tumor microenvironment. Following knockdown of WHSC1 in vitro, we observed a downregulation of AR and KLK2, suggesting that inhibition of WHSC1 in C42 cells limits AR signaling, corroborating previous studies showing increased immune infiltration in the tumor following ADT. Previous studies neither investigate nor identify the tumor-resident immune pathways that define the interface between prostate tumors and immune system. We demonstrated via RNASeq and flow cytometry analysis that silencing of WHSC1 increased WIC expression on prostate cancer cells, augmenting the levels of MHC-bound antigen on the cell's surface. Consequentially, we observed increased T cell infiltration in grafted tumors following pharmacological inhibition of WHSC1 with MCTP39, which correlated with reduced tumor growth. In our study we used immunocompetent C57B/6 mice grafted with syngeneic TRAMP C2 cells and found reduced tumor weight, increased T cell infiltration and increased MHC expression in treated tumors. The TRAMP C2 cells were shown to be a reliable subcutaneous in vivo model for studying the behavior of the immune system in prostate cancer in response to therapy (Poggio et al., Cell. 2019; 177(2):414-27 e13. Epub Apr. 6, 2019. doi: 10.1016/j.ce11.2019.02.016. PubMed PMID: 30951669; PMCID: PMC6499401; Mikyskova et al., Int J Oncol. 2016; 48(3):953-64. Epub Jan. 1, 2016. doi: 10.3892/ijo.2015.3314. PubMed PMID: 26718011; PMCID: PMC4750542; Pai et al., Immunity. 2019; 50(2):477-92 e8. Epub Feb. 10, 2019. doi: 10.1016/j.immuni. 2019.01.006. PubMed PMID: 30737146; PMCID: PMC6886475). We indeed demonstrate changes in immune infiltrate and WIC expression consistent with the induction of an anti-tumor immune response. Furthermore, we tested whether a similar magnitude in tumor reduction was observed in immunocompromised mice and found only a limited and non-significant effect of MCTP39 in delaying tumor growth. While this suggests that the immune system plays a certain role in mediating the observed antitumor effect, more studies are needed to pinpoint the exact immune population(s) responsible for this effect. Since NSG mice lack of T, B, NK and complement cells, it is possible that the reduction in tumor growth observed in immunocompetent mice is the result of the coordinated action of multiple immune cell types. To this point, we demonstrate that following WHSC1 inhibition there is higher antigen presentation in vitro and higher T cell infiltration in vivo. This suggests that T cells are the most likely driver of the anti-tumor response that we observed.

Mechanistically, we demonstrate a close relationship between WHSC1 and DNMT1 expression, suggesting a role for WHSC1 in maintaining the DNA methylation status in PCa. While our results do not directly indicate which DNA methyltransferase enzyme is the main driver for the changes we observed, RNASeq data point to DNMT1, since its expression levels are reduced following WHSC1 knockdown and DNMT1 is located within H3K36me2/3 loci.

Here we offer a novel function for WHSC1/NSD2 as key regulator of tumor-resident immune pathways and WHSC1 pharmacological inhibition has the potentials to act as a potent adjuvant for combination immunotherapy in prostate cancer.

### EXAMPLE 2

This describes a prophetic example to define a comprehensive profile of chromatin modifications driven by WHSC1 in PCa. WHSC1 is a ubiquitous histone methyltransferase. Our data using published ChIPSeq and the ATACSeq data strongly suggest that WHSC1 alters the chromatin landscape and that genes in the APM pathway reside within H3K36me3/me2 loci. By obtaining a comprehensive map of the H3K36me2/me2 loci regulated by WHSC1, a bona-fide picture can be acquired of the epigenetic mechanisms by which WHSC1 directly affects cellular pathways, including antigen processing and presentation and DNA damage response. This can define the direct epigenetic changes driven by WHSC1 in castrate-resistant prostate cancer (CRPC).

Methods: ChIPSeq: Standard X-ChIP can be performed on WT or engineered C42, PC3 and DU145 cells prior to sequencing, as we previously described (Battaglia et al., Carcinogenesis, 2010. 31(9): p. 1650-60), in duplicate for the following conditions: 1) WT cells, 2) WHSC1ko 3) WT cells+IFNg 4) WHSC1ko+IFNg, 5) WT cells+MCTP39 and 6) WT cells+MCTP39+IFNg. Briefly, cells can be cross-linked with 1% formaldehyde for 8 min at 37° C. and reaction can be stopped using 0.125M glycine. Cells can be washed and collected in cold PBS, pellet resuspended in lml lysis buffer and sonicated. To avoid over/under-sonication we can perform quality checks every cycle (5 minutes) up to 40 minutes to ensure a fragment size within 200-600 bp. Sonicated samples can be incubated with IgG, H3K36me3 and H3K36me2 antibody over night at 4 C, the following day samples can be washed and eluted. DNA can be extracted using columns. Library prep and sequencing can be done by standard methods. Raw sequence reads can be aligned to reference genome using Bowtie2 and peaks called with Model-based Analysis of ChIPSeq (MACS). Peaks can be annotated using the ChIPpeakAnno R package to identify the nearest gene up to a 5 kb span.

RNASeq: Cells lines can be divided in the groups described above, in triplicate. RNA extraction, library prep and sequencing can be done by standard methods. Raw reads can be processed with Spliced Transcripts Alignment to a Reference (STAR) aligner and aligned to the hg38 human genome. Gene level counts can be used for QC prior DEG analysis. The limma R package can be used to identify DEGs across conditions and cell lines. Functional enrichment analysis can be performed with gene count data using Gene Set Variation Analysis (GSVA). RNASeq data can be integrated with ChIPSeq data using Binding and Expression Target Analysis (BETA) to infer the causative regulatory function of each epigenetic modification on gene transcription per each cell line and condition.

A prophetic example to profile changes in the spatial relationship between chromatin regions driven by WHSC1 is provided. Transcription does not occur in a linear manner, studies have demonstrated that long-range cis- and trans-elements influence local gene transcription. Hi-C is a chromatin conformation capture method to identify chromosomal interactions within and between chromosomes. We can perform it in three androgen CRPC cell lines to define 1) how WHSC1 alters chromatin spatial relationships in CRPC, and 2) the contribution of WHSC1 in 3D chromosomal changes. Thus, the 3D chromosomal structure dictated by WHSC1 in CRPC can be defined.

Methods: Sample preparation, DNA isolation: Hi-C allows to identify local (cis) and distal (trans) chromosomal interactions spanning all genomic regions (all vs. all). Cell lines, 100×10⁶cells/group/cell line (described above) can be treated with low concentration of Accutase prior crosslinking with a 1% solution of formaldehyde in serum-free media, followed by 0.125M glycine to stop crosslinking. Cells can be digested with lysis buffer and protease inhibitor and homogenized with a pestle prior centrifugation to isolate pellets. After resuspending the pellet in ice cold NEBuffer, proteins that were not crosslinked with the DNA can be degraded adding 1% SDS buffer and incubating samples at 65 C for 10minutes. Triton-X100 can be added to quench the SDS and samples digested with HindIII (400U) overnight at 37 C. DNA ends can be marked with dNTPs and biotin-14-dCTP followed by blunt end ligation using 50U of T4 ligase and incubated 4 hours at 16 C. Following ligation, proteins can be degraded using proteinase K at 65 C for 2 hours and DNA extracted using columns. To degrade any potential RNA, RNase A can be added and samples incubated at 37 C for 15 minutes. At this step, sample and quality can be checked via PCR using published primer sequences to evaluate the presence of Nhel digestion site, (introduced by fill-in and ligation of the HindIII). Biotinylated-14-dCTP can be removed from the unligated ends with the exonuclease activity of the T4 enzyme and DNA isolated with phenol-chloroform and resuspended in DNase-free water. Shearing size can be evaluated using agarose gel following sonication. To avoid over/under-sonication we can perform quality checks every cycle (5 minutes) to ensure a fragment size within 300-500 bp. As last step prior library prep, biotin-tagged Hi-C DNA is pulled down using Streptavin magnetic beads through cycles of washes and purification on magnetic rack prior collecting the samples in ligation buffer. Library prep and sequencing can be carried out by standard methods.

Data analysis: Raw fastq files containing unmapped reads can be processed through two Hi-C pipelines to generate paired-reads mapped files (using HiCUP), followed by estimation of DNA-DNA contact regions and map generation. Briefly, we can use HiCUP to align raw reads using Bowtie2 prior filtering out paired reads as result of experimental artifact (i.e. fragment ligated to itself, tags mapping to adjacent restriction fragments that have re-ligated in the same orientation as found in the genome, tag length off size, tags spanning several restriction fragments) and removing duplicated tags as result of PCR duplicates. Lastly, we can remove the remaining invalid pairs by using a computationally digested reference genome (with HindIII). The remaining reads passing quality filter (MAPQ>30) can be used to generate bam files for HIFI. HIFI estimates interaction frequencies between genomic regions using a dynamic binning method (Christopher et al., bioRxiv, 2019. 1(17)), hence allowing to identify topologically associated domains (TADs) at high resolution. We can then compare interaction matrices with HiCdat to identify enrichment/depletion of interaction frequencies across samples.

Data integration: RNASeq and ChIPSeq data can be integrated. Hi-C data can be modeled considering the location of the cis/trans interactions, which can be annotated based on the distance from the nearest gene and the presence of enhancer/silencer regions. Regions marked for differential chromosomal interactions can be fed to a generalized linear model using gene expression values as outcome and RNASeq expression as continuous predictor variable. Results can be controlled for family wise error rate (FWER) using false discovery rate (FDR) of 5%. Causal relationships between histone modifications and gene transcription can be integrated with Hi-C based on genomic location with the hypothesis that WHSC1-mediated histone modifications promote/limit 3D chromosomal interactions.

From these studies, WHSC1 inhibition can define an epigenetic signature reflecting higher potential immunogenicity of tumor cells, reflected by changes in both cis-and trans-regulatory elements relationships with histone methylation and 3D conformation.

Since we can inhibit WHSC1 via CRISPR/Cas and MCTP39, we might observe slightly different, albeit overlapping, results due to off targets effect of the drug. Should this be the case we can consider results common between the two interventions. Should we identify no differences in chromatin status H3K36me2/me3 after altering WHSC1 levels or function, it can be an indication that the epigenetic modifications responsible for the changes in HLA expression are not in the panel we investigated and/or there are indirect secondary effects that alter gene transcription. Should this be the case, based on preliminary indications that WHSC1 regulates DNMT1 expression, we can analyze changes in the methylome upon WHSC1 inhibition via Whole Genome Bisulfite Sequencing (WGBS).

### EXAMPLE 3

This is a prophetic example to define in vivo the heterogeneity of chromatin alteration that define cellular phenotype in response to checkpoint blockade and WHSC1 inhibition. WHSC1 knock down significantly downregulates CDK12, BRCA1, BRCA2 and DNA repair genes (FIG. 22), suggesting that WHSC1 inhibition might promote a transcriptional signature favoring response to anti-PD1. Preliminary in vivo data also show MCTP39 treatment may increase intratumoral CD8⁺PD1⁺ T cells. Overall, we investigated if the combination of PD1 and WHSC1 inhibition can have an additive anti-tumor effect, with WHSC1 inhibition epigenetically priming the tumor for a potent T cell response, and PD1 inhibition silencing T cells inhibitory signals while potentiating T cell priming by DCs. The objective is to evaluate the therapeutic effect of WHSC1 and anti-PD1 inhibition.

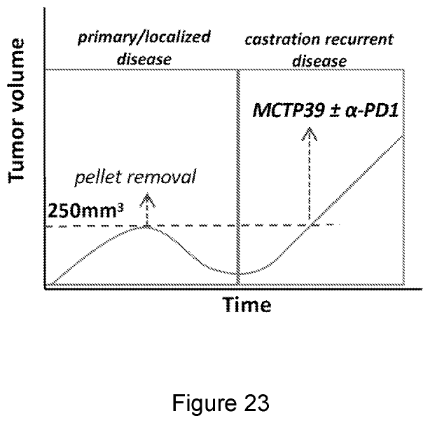

In vivo treatments: C57B/6 mice (n=20/group (see below), 10-12 weeks old) can be surgically castrated and two days later testosterone pellet (12.5 mg/mouse) can be implanted subcutaneously in the upper back. After 48 hours mice can be grafted orthotopically with TRAMP C2 or PTEN^{-/-}/RB^{-/-} cells in Matrigel (1.5×10⁶/100μ). Briefly, minimally invasive orthotopic grafts can be done by ultrasound guided injection to target the anterior prostate. This procedure requires no surgery and causes no local inflammation. Injections can be performed under imaging. Tumor size can be monitored weekly via Mill imaging. When tumors reach 250mm³ (FIG. 23), pellets can be removed, and mice randomized into treatment groups depending upon WHSC1 knockout status (WT or WHSC1ko): 1) WT+vehicle, 2) WT+MCTP39, 3) WT+anti-PD1, 4) WT+anti-PD1+MCTP39, 5) WHSC1ko+vehicle and 6) WHSC1ko+anti-PD1. When tumors reach 250 mm³ (FIG. 23), mice can receive 200 mg anti-PD1 or MCTP39 10 mg/kg IP every other day (MWF) for 10 weeks. Mice can be imaged once/week for a total of 12 weeks post treatment. Endpoint is defined as the presence of a mass of more than 2 cm in any dimension or an increase of more than 20% in body weight. Mice whose tumors do not reach maximum size can be sacrificed after 12 weeks of treatment. Powering the in-vivo study for size comparisons, at 20 mice/group, there is 80% power to detect a 91 mm³ difference in tumor volume at a significance level of 0.05. We based this comparison on reference curves presented in other studies of C2 cells (Babiarova et al., J Immunother, 2012. 35(6): p. 478-87, Chang et al., J Urol, 2010. 183(4): p. 1611-8). This is based on the two-sample t-test, which should be conservative for the ANOVA analysis across the study groups. In variations of this example, instead of anti-PD1, inhibitors of PARP or TK or other targeted therapies can also be used.

Single cell (sc) isolation: At endpoint, tumors can be resected and processed. Briefly, tumors can be minced with a scalpel followed by collagenase digestion at 37 C for one hour. The enzymatic reaction can be stopped by adding media with 8% FBS, P/S and EDTA following filtering through a 70 μm strainer at 4 C; cells can be washed, spun down and collected. Tumor and immune cells can be separated using CD45 beads.

scATACSeq: Nuclei isolation using at least 100 k cells/sample, incubation with the transposase Tn5 enzyme, library prep and sequencing reactions can be performed by standard methods. Raw files can be processed through the CellRanger ATAC pipeline (10× Genomics) followed by analysis using the Signac R Package, a module within the Seurat suite, designed to explore and integrate highly dimensional single cell data. Briefly, following preprocessing and QC, a two-step normalization can be done (Term frequency-inverse document frequency (TF-IDF)) across cells and peaks, followed by feature selection to optimize dimensionality reduction via single value decomposition (SVD). The resulting low dimensional data can be evaluated via Uniform Manifold Approximation and Projection (UMAP), to identify spatial relationship between groups/cells, peaks can be annotated to the nearest gene and superimposed over the UMAP map and used to label cells with similar phenotype. At this step data are ready for integration with scRNASeq data (described below), which offer a functional interpretation into the scATACSeq results.

scRNASeq: Isolated tumor and immune cells can also be subject to scRNASeq to integrate the transcriptional dimension onto the single cell chromatin conformation data (tumor) and profile the resident immune moieties (immune cells). Briefly, library preparation from single cell suspension and sequencing can be performed by standard methods and raw data processed using 10X' s Cell Ranger tool. Differentially expressed genes can be identified using DESeq2. scRNASeq then processed using Seurat. Briefly, after preprocessing and QC, single cell data are normalized and highly variable features (genes) across cells are identified prior scaling the data for clustering and dimensionality reductions (UMAP) to identify tumor cell cluster reflecting the potential heterogeneity in therapeutic response. For immune cells (CD45⁺ fraction), a similar approach using Seurat/UMAP can be used to separate cell clusters and identify highly expressed cell markers per each cluster. We can also use SingleR, an algorithm designed to identify immune cell population from scRNASeq, to define the abundance and type of immune moieties. Significant differences between treatment conditions can be evaluated with ANOVA at a significance level of 0.05, with data transformation applied as needed based on population abundance.

Data integration: Processed scRNASeq data can be embedded with scATACSeq data by calculating anchors. First the internal structure of scATACSeq data is reduced using Latent Semantic Indexing (LSI) implementing TF-IDF and SVD (see above); once anchors are identified, cell type labels can be transferred across samples. Data can then be co-embedded into the same low dimensional space. Overall, this data integration captures the epigenetic and transcriptional heterogeneity as consequence of WHSC1 inhibition and anti-PD1 therapy. Pathway enrichment analysis can be integrated to layer functional annotation onto each cluster and interpret the heterogeneity of the response to therapy. This could help identifying potential novel driver that conferred therapeutic resistance to a subset of tumor cells. Gene expression and peak intensity can be fed to a generalized mixed model with random mouse effect to compound for data source and potential in vivo sample heterogeneity. We can control for FWER using a false discovery rate (FDR) of 5%.

The combined treatment can have an additive or synergistic, anti-tumor effect, defined by 1) higher infiltrating cytotoxic TILs and 2) the presence of epigenetic and transcriptional signature reflecting activation of tumor immune pathways.

Alternatively Dox-inducible shWHSC1 knockdown cells can be generated to allow WHSC1 depletion only in the castration recurrent phase in combination with anti-PD1. Should we observe no differences in tumor growth following WHSC1 inhibition, we can leverage scRNASeq and scATACSeq data to identify compensatory mechanisms, and potential targets, that overtook the anti-cancer effects of WHSC1 inhibition.

While the invention has been described through embodiments, routine modifications to the disclosure here will be apparent to those skilled in the art. Such modifications are intended to be within the scope of this disclosure.

What is claimed is:

1. A method for treatment of cancer, comprising inhibiting the expression, function, or activity of WHSC1 in an individual in need of treatment, and administering to the individual an inhibitor PARP or immune based therapy.

2. The method of claim 1, wherein the cancer is a tumor.

3. The method of claim 1, comprising administering to the individual an inhibitor of WHSC1 in combination with an inhibitor PARP or in combination with immune checkpoint inhibitor.

4. The method of claim 3, wherein the inhibitor of WHSC1 is MCTP-39.

5. The method of claim 1, wherein the cancer is selected from the group consisting of prostate cancer, pancreatic cancer, lung cancer, melanoma of the skin, kidney cancer, bladder cancer, liver cancer, colon cancer, head and neck cancers, breast cancer, ovarian cancer, cervical cancer, Hodgkin lymphoma, and urinary tract cancers.

6. The method of claim 3, wherein the cancer is prostate cancer.

7. The method of claim 1, wherein the immune based therapy is small molecule inhibitors, monoclonal antibodies, cancer vaccines, and/or T-cell based therapies, NK-cell based therapy.

8. The method of claim 1, wherein the immune based therapy is administration of checkpoint inhibitor.

9. The method of claim 6, wherein the checkpoint inhibitor is directed against immune checkpoints PD-1, PD-L1, CTLA-4, LAG-3, or Tim-1.

10. The method of claim 9, wherein the checkpoint inhibitor is directed against PD-1.

11. The method of claim 8, wherein the checkpoint inhibitor is selected from the group consisting of nivolumab, pembrolizumab, cemiplimab, pidilizumab, PDR001, MEDI4736/duralumab, and ABBVI-181, atezolizumab, durvalumab, avelumab, TSR-033, or TSR-022.

12. The method of claim 1, wherein the PARP inhibitor is olaparib, rucaparib, niraparib, talazoparib, veliparib, pamiparib, CEP9722, E7016, or 3-aminobenzamide.

13. The method of claim 12, wherein the WHSC1 expression is inhibited by using CRISPR.

14. The method of claim 3, wherein the inhibitor of WHSC1 and PARP are administered concurrently or sequentially, by the same routes or different routes, over the same period of time or different periods of time.

15. The method of claim 3, wherein the inhibitor of WHSC1 and immune checkpoint inhibitor are administered concurrently or sequentially, by the same routes or different routes, over the same period of time or different periods of time.

16. A composition comprising i) an inhibitor of WHSC1 and ii) an inhibitor of PARP or an immune checkpoint inhibitor.

17. The composition of claim 13, wherein the WHSC1 inhibitor is MCTP-39.
